(19) United States
(12) Patent Application Publication
(10) Pub. No.: US 2023/0337955 A1
(43) Pub. Date: Oct. 26, 2023

(54) COMPENSATING FOR HUMAN-MACHINE INTERFACE DISRUPTIONS

(71) Applicant: BATTELLE MEMORIAL INSTITUTE, Columbus, OH (US)

(72) Inventors: Laura L. Aume, Columbus, OH (US); Sam Colachis, Columbus, OH (US); Collin F. Dunlap, Columbus, OH (US); David A. Friedenberg, Columbus, OH (US); Jordan L. Vasko, Columbus, OH (US)

(21) Appl. No.: 17/927,076

(22) PCT Filed: May 24, 2021
(86) PCT No.: PCT/US2021/033866

§ 371 Date: Nov. 22, 2022

Related U.S. Application Data
(60) Provisional application No. 63/029,333, filed on May 22, 2020.

(51) Int. Cl. A61B 5/276; G06F 3/01; A61B 5/293; A61B 5/00

(52) U.S. Cl. CPC A61B 5/276; G06F 3/015; A61B 5/293; A61B 5/7203; A61B 5/7221; A61B 5/7267; A61B 2562/04; A61B 2562/028; A61B 2562/0209

(57) ABSTRACT

The present disclosure provides systems and processes for compensating disruptions in a brain-machine interface (BMI). Briefly described, the systems and processes detect and compensate for transient disruptions, reversible disruptions, irreversible compensable disruptions, or irreversible non-compensable disruptions.

### STATEMENT REGARDING FEDERALLY SPONSORED RESEARCH OR DEVELOPMENT

This invention was made with government support under Defense Advanced Research Projects Agency (DARPA) Grant Number DARPA-PA-18-02-04-INI-FP-006 awarded by the Department of Defense. The government has certain rights in the invention.

### BACKGROUND

### Field of the Disclosure

The present disclosure relates generally to brain-machine interfaces and, more particularly, to compensating for disruptions in brain-machine interfaces.

### Description of Related Art

A brain-machine interface (BMI) is a type of human-machine interface that records and translates neural activity into signals. For instance, an individual with motor or sensory impairment can use a BMI to translate neural activity into commands that control external hardware, such as a robotic appendage. An intracortical microelectrode array (MEA) enables the BMI to collect neural information. BMIs, including associated MEAs, have been known to experience failures, which have been categorized as biological, material, or mechanical.

### DISCLOSURE OF INVENTION

The present disclosure provides systems and processes that compensate for disruptions in a brain-machine interface (BMI). Briefly described, the systems and processes detect and compensate for transient disruptions, reversible disruptions, irreversible compensable disruptions, irreversible non-compensable disruptions, or combinations thereof. In some embodiments, these disruption categories are used in conjunction with causal categories, such as, for example, biological disruptions, material disruptions, or mechanical disruptions.

Other systems, devices, processes, features, and advantages will be or become apparent to one with skill in the art upon examination of the following drawings and detailed description. It is intended that all such additional systems, processes, features, and advantages included within this description, be within the scope of the present disclosure, and be protected by the accompanying claims. In this regard, the components, processes, and systems herein can be combined in any order, and in any configuration.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a flowchart showing an embodiment of a sensor compensation process.

FIG. 2 is a flowchart showing an embodiment of a monitoring process as shown in FIG. 1.

FIG. 3 is a flowchart showing an embodiment of a disruption determining process as shown in FIG. 1.

FIG. 4 is a flowchart showing another embodiment of a disruption determining process as shown in FIG. 1.

FIG. 5 is a flowchart showing an embodiment of a mitigation process for the disruptions determined in FIG. 4.

FIG. 6 is a flowchart showing an embodiment of a transient disruption determining process as shown in FIG. 4.

FIG. 7 is a flowchart showing an embodiment of a reparable disruption determining process as shown in FIG. 4.

FIGS. 8A and 8B are flowcharts showing an embodiment of an irreversible compensable disruption determining process as shown in FIG. 4.

FIGS. 9A and 9B are flowcharts showing an embodiment of an irreversible non-compensable disruption determining process as shown in FIG. 4.

FIG. 10 is a flowchart showing an embodiment of a transient disruption mitigation process for the transient disruptions of FIG. 6

FIG. 11 is a flowchart showing an embodiment of a reparable disruption mitigation process for the reparable disruptions of FIG. 7

FIG. 12 is a flowchart showing an embodiment of an irreversible compensable disruption mitigation process for the irreversible compensable disruptions of FIGS. 8A and 8B.

FIG. 13 is a flowchart showing an embodiment of an irreversible non-compensable disruption mitigation process for the irreversible non-compensable disruptions of FIGS. 9A and 9B.

FIGS. 14A, 14B, and 14C (collectively, “FIG. 14”) show classification of common MEA signal disruptions and applicable compensatory strategies.

### MODES FOR CARRYING OUT THE INVENTION

Conventionally, failures in brain-machine interfaces (BMIs) (e.g., in the BMI, microelectrode array (MEA), or combination thereof) have been categorized as biological, material, or mechanical. In other words, the failures are categorized on the basis of the cause of the disruption. However, these causal categories provide insufficient information to compensate for the disruption caused by the failure. The improvements to the brain-machine interfaces discussed in detail herein may also be applied similarly to any human-machine interface that for implanting on a person.

To mitigate this deficiency, the present disclosure provides systems and processes for determining the impact of disruptions on brain-machine interfaces (BMIs) and compensating for those disruptions. Briefly described, the systems and processes determine whether or not a disruption is a transient disruption, reversible disruption, irreversible compensable disruption, irreversible non-compensable disruption, or combinations thereof, all of which represent the impact of the disruptions (rather than the cause of the disruptions). Thereafter, the systems and processes compensate for these impacts. In some embodiments, these disruption categories are used in conjunction with causal categories, such as, for example, biological disruptions, material disruptions, or mechanical disruptions.

Having provided a broad technical solution to a technical problem, reference is now made in detail to the description of the embodiments as illustrated in the drawings. While several embodiments are described in connection with these drawings, there is no intent to limit the disclosure to the embodiment or embodiments disclosed herein. On the contrary, the intent is to cover all alternatives, modifications, and equivalents.

### SECTION 1: Introduction

Brain machine interface (BMI) systems have been proposed as assistive devices to restore, replace, or augment lost motor function to people with paralysis. These neural interface systems record and interpret brain signals, enabling control of an effector device through modulation of neural activity. Noninvasive neural recording techniques including electroencephalography (EEG), functional near-infrared spectroscopy (fNIRS), and functional magnetic resonance imaging (fMRI) can function as sensors for BMIs. However, these systems lack spatiotemporal resolution or information transfer capacity required for accurate and intuitive control of high degree-of-freedom (DOF) effectors. Unlike such recording techniques, MEAs are known that can provide adequate spatiotemporal resolution/information transfer capacity required for BMI sensors. However, recording disruptions that affect MEA/BMI performance can limit their usefulness.

A significant barrier to the widespread adoption of intracortical neural interfaces as assistive devices is the limited lifetime of the recording array. For instance, intracortical MEA failure modes suggest that device failure can occur within a relatively short time span, e.g., within a year of implantation, at least in non-human primates (NHPs). Even in the absence of acute failures, MEAs may exhibit less than a decade of useful life due to persistent decline in recording quality over time. Moreover, the longevity of MEAs in humans is still unresolved. For instance, clinical trials that have investigated the functionality of intracortical BMIs beyond four years post-implant have reported sustained usability.

However, chronic declines in signal quality over time have been observed in human trials. These declines are sometimes persistent, progressive and irreversible because the underlying disruptions affect the tissue-electrode interface (e.g., local scarring or meningeal encapsulation) or implanted hardware (e.g., electrode insulation deterioration). Such signal disruptions require neurosurgical intervention for hardware exchange to completely resolve. Device replacement to improve performance or evaluate the hardware for damage can be both prohibitively costly and risky, involving potential for cortical tissue injury during explant and reimplant as well as risk for infection, hemorrhage, and adverse reaction to anesthesia. Identifying chronic signal disruption types that may be amenable to nonsurgical remediation could potentially extend the longevity and enhance the attractiveness of intracortical BMIs as assistive neurotechnology.

Another challenge affecting practical usability of intracortical neural interfaces is dynamic neural signal drift and other transient disruptions. For example, the presence of an object in a neuroprosthetic reach and grasp task may transiently affect neural population firing rates and complicate decoding of intended grip states. Additionally, micromovements of the MEA and cognitive fatigue can impact how neural features are represented across channels over time. A common technique used with humans to mitigate these signal instabilities is to train intracortical BMI algorithms de novo on a daily basis. However, transient disruptions can decrease BMI performance to chance levels in as little as 30 minutes. This effectively renders the interface useless until the disruption is resolved or the decoder is recalibrated. Recalibration prolongs set up time. However, set up time should be minimized as much as possible to aid in the convenience to candidate BMI users.

Other transient disruptions, e.g., FES stimulation artifact, introduce noise into a recording that must be removed to avoid temporary loss of control when operating a physical effector like a grip orthotic. Therefore, recognizing and accounting for transient signal instabilities are important ways to improve convenience, safety, and eventual adoption of BMI systems. Consequently, detecting and mitigating MEA signal disruptions on both chronic and acute time scales are important, open challenges for the field.

The root causes of failures can be sorted into three main categories: biological, material, or mechanical. This organization is convenient for grouping failures with similar underlying causes, and may suggest improvements. For example, mechanical design considerations should take into account the observation that electrodes should be strong enough to withstand physical forces exerted during cortical insertion, but also sufficiently compliant to minimize micromotion-induced strain on surrounding tissue.

Biological and material design constraints respectively dictate that MEA devices should not elicit a foreign body response and should be resistant to electrode corrosion and insulation deterioration. While the neurotechnology field is advancing, even the best neural implants are subject to a range of potential disruptions that affect MEA signals and limit BMI system performance.

An alternative approach to counteract signal deterioration is the development of algorithmic methods to monitor and compensate for disruptions. One benefit of this approach is its potentially short timeline for development, deployment, and impact. In contrast to the extensive and time-consuming regulatory approval process required for hardware modifications, software can be rapidly implemented and upgraded, conferring immediate benefits to the user. Another advantage of this approach is its inherent flexibility and customization potential. Software can be made to adapt to chronic changes in signal characteristics and tailored to specific users or disruption processes.

When designing algorithmic strategies to mitigate signal disruptions, the underlying cause of a disruption becomes secondary in importance to its impact on recorded signals. With this shift in perspective, it becomes evident that the categorization of disruptions as biological, material, or mechanical can be augmented to include temporal characteristics of the disruption and a sense of whether and how the signal is recoverable. Within each of these three causal categories, disruptions may have vastly different consequences on signal quality. For example, neuroinflammation, glial scarring, and neurophysiological state changes are all of biologic origin but likely impact distinct attributes and time scales of recorded signals.

As such, aspects herein provide a set of disruption categories that describe the changes of recorded signals and the amenability of those changes to algorithmic compensation. Aspects herein further classify commonly observed disruptions of MEA recordings into one of four groups according to the following definitions:

Transient Disruptions interfere with recordings on the time scale of hours or less and may resolve spontaneously. However, recorded signals do not necessarily revert to a previous state following a transient disruption.

Reparable Disruptions cause persistent interference in recordings that typically does not spontaneously resolve. Good signal quality can be restored with a targeted intervention that addresses the root cause.

Irreversible Compensable Disruptions cause persistent or progressive reduction in signal quality. While the underlying cause cannot be remedied, the effects may be compensated for algorithmically.

Irreversible Non-Compensable Disruptions cause persistent or progressive reduction in signal quality, cannot be remedied by fixing the root cause, and are not amenable to algorithmic compensation. These disruptions indicate severe failures that may render the interface inoperable.

Assigning disruptions into these categories is useful because each category aligns closely with strategies to detect and correct signal disruptions. For instance, adaptive decoding algorithms can be utilized to compensate for the acute shifts in neural recordings caused by transient disruptions. Likewise, algorithms that monitor longitudinal signal quality can detect reparable disruptions such as faulty connections or external cable damage and may provide clues that a user is fighting a systemic infection that requires antibiotics. Irreversible, compensable disruptions, such as the formation of a glial scar or electrode insulation cracking, may be overcome by optimizing neural decoding features in affected channels. Irreversible, non-compensable disruptions such as meningeal encapsulation and ejection of the MEA from the cortex result in widespread signal loss that cannot be recovered with algorithmic strategies. Notably, these categories are not required to be entirely mutually exclusive. For instance, in an example embodiment, some disruptions may fall in more than one category based on severity. Nonetheless, the broad categorization is a useful construct for organizing disruptions by performance impact and potential for remediation.

MEA signal disruptions of biological, material, and mechanical etiologies are discussed in greater detail herein. Moreover, aspects herein demonstrate applications of the proposed expanded classification method. Moreover, as will be described in greater detail herein, mitigation strategies are provided, which are appropriate to each of the newly introduced categories.

The signal disruptions may be found using metrics such as impedance values, MEA signal values (root-mean-square voltage (V_{RMS}) and peak-to-peak voltage (Vₚₚ)), identified units (defined as one or more neurons with spike waveforms that can be clustered using wavelet transform features or other waveform characteristics, firing rate (FR), signal-to-noise ratio (SNR), channel correlation (calculated by first correlating raw voltage waveforms for each channel with all other channels), or combinations thereof while the patient is at rest. Some of the disruptions use the signal values described above during a motor imagery task (e.g., a four-step motor function that includes an index finger flexion, an index finger extension, a wrist extension, followed by a wrist flexion). These metrics provide insight to the chronic material and biological processes that affect signal quality.

Over time, there is normally thought to be an exponential decline in impedance immediately after implantation of an MEA (e.g., a rapid decline in impedance has been observed after a few weeks). However, recent tests have shown that there is an initial rise in impedance for the first few weeks after implantation, so it is suggested that external biological factors can affect (even dominate) the impedance including resolving post-implant edema and inflammation or MEA material processes such as water absorption and infiltration.

Peak to peak voltage experiences a decrease within the first moths after implantation (e.g., a 54% decline in average Vpp with a half-life of 68 days was observed). Thus, there is a suggestion of gradual deterioration of signal quality over time.

A bottom-up method, a top-down method, or both may be used to identify disruption cases. The bottom-up method includes analyzing signals to retrospectively identify cases of signal disruptions and then classifying the disruptions by studying the signal and documented evidence. On the other hand, the top-down method includes analyzing a chronic signal to identify trends associated with array failure modes.

### SECTION 2: Biological Disruptions

Adverse biological reactions to neural implants are well characterized. To date, there has been no functional intracortical multi-electrode recording device that completely avoids the biological responses to implantation that preclude long-term neural recordings. As such, chronic biological reactions are expected to affect signal acquisition, with various biological sources of acute recording disruptions that can be detrimental to decoding accuracy.

### SECTION 2.1: Blood Brain Barrier (BBB) Damage

Electrode implantation causes trauma to cortical tissues and directly damages the blood-brain barrier. Penetrating electrodes displace local tissue and cause minor cortical tearing in addition to rupturing, severing, and dragging of the microvasculature. Even though the arrays are carefully placed to avoid major vessel trauma during implantation, MEAs inevitably cause microvascular damage because individualized electrode placement around microvessels is not possible. Implantation in the human cortex can cause microhemorrhages around electrode tracks and petechial hemorrhages below electrode tips. BBB disruption, evidenced by local increases of ferritin, immunoglobulin, and albumin at the electrode-tissue interface, persists throughout the entire implant duration and is associated with poor recording performance. BBB disruption degrades recording quality through several mechanisms.

First, the damaged vasculature enables infiltration of proinflammatory macrophages and myeloid cells at the implant site. These cells produce cytokines that promote neuroinflammation, enhance BBB permeability, and create a feedback loop that propagates chronic inflammation, neurodegeneration, and signal deterioration. Secondly, loss of the BBB facilitates plasma protein leakage into the peri-electrode space, contributing to astroglia and microglia activation, further amplifying neuroinflammation. Erythrocyte infiltration and degradation following microhemorrhages at the implant interface increases free iron levels, which in turn promotes local oxidative stress. Lastly, damaged vasculature allows for an unregulated influx of molecules around the array that can disrupt local ionic gradients and synaptic stability, ultimately resulting in variable neuronal responses.

Signal Disruptions Due to BBB Damage

Transient Disruptions: Acute neuroinflammation and homeostatic imbalances cause acute firing rate modulations of neurons recorded by the array as well as changes in background biological noise. These biological responses decrease recording consistency, which can negatively impact BMI decoder performance. Resolution of acute neuroinflammation can reverse these signal changes.

Irreversible Compensable Disruption: Chronic inflammation is associated with minor loss of neurons around the array, resulting in a decrease of available information in the MEA recording. Neurodegenerative states such as these are associated with chronic, slowly progressive increases in neural response variability, dropout of previously recorded units, and decline in signal to noise ratio of recorded signals.

### SECTION 2.2: Tissue Encapsulation

Following device implantation, microglia and astrocytes are activated and recruited to the electrode interface where they form a sheath around electrodes. The extent of glial scarring is variable, and selective electrode encapsulation can occur for neighboring recording sites in the same array. Such inconsistencies could be due to variations in local tissue and microvascular damage during implantation. Electrodes surrounded by increased densities of non-neuronal cells, including microglia and astrocytes, tend to acquire lower quality signals. Furthermore, MEAs are susceptible to fibrous encapsulation that can cause gross array movement, chronic recording instability, and widespread signal loss. In this regard, there are several of the mechanisms by which glial scarring and fibrous encapsulation can affect recorded signals.

Glial scarring is most likely to disrupt recordings during acute, post-implant scar formation and tissue stabilization around the implant. This process is commonly identified as the cause for the substantial increase in electrode impedance typically seen within the first weeks after implantation. Heightened impedance with scar formation suggests that the scar electrically insulates the implanted device and restricts current flow. The insulating role of the glial scar demonstrates that the glial sheath inhibits molecular diffusion. In addition, scar formation may influence synaptic transmission and modulate surrounding cellular and neuronal population activity, altering MEA signal characteristics as the scar forms. Changes in glial scar morphology post-implant, highlight the potential for dynamic changes in the MEA recording environment over this timeframe. Nevertheless, scar stabilization and chronic decreases in recording quality are not temporally aligned, necessitating the involvement of other failure mechanisms in loss of signal quality.

Activated glial cells may contribute to chronic signal disruptions by producing proinflammatory cytokines that can lead to neurodegeneration. Indeed, high levels of activated glial cells are associated with neuronal loss adjacent to electrodes, which is likely a result of neurotoxic inflammation. Furthermore, these scars are known to create a local inhibitory environment that impedes axon regeneration. Irreversible neuronal loss decreases the signal-to-noise ratio (SNR) of recorded signals and causes dropout of previously recorded units. However, a recent study illustrated that glial scarring has less of an impact on signal quality, impedance identified units, and SNR than previously thought.

Meningeal encapsulation can be a significant failure mode of intracortical electrodes, thus affecting recording quality. Also, it is possible that time course and the effects of parenchymal encapsulation can affect recording quality. In this regard, meningeal encapsulation and extrusion of intracortical arrays can be considered a chronic failure mode of NHP MEAs. Encapsulation occurs when meningeal cells migrate down the implant from the cortical surface and form a capsule that conforms to the implant and thickens over time. The tissue capsule exerts mechanical forces that can ultimately eject the device from the cortex. Excessive local meningeal proliferation can also result in a downward pressure that causes indentation of the cortical surface. In either case, movement of the array changes the depth of the recording sites in the cortex and chronically disrupts signal stability.

Signal Disruptions Due to Tissue Encapsulation:

Irreversible Compensable Disruptions: Scar formation and stabilization can be associated with increased impedance, reduction in signal amplitudes, and decreased signal to noise ratio (SNR) due to electrode encapsulation and neuronal loss. Fluctuations in scar morphology and local neuronal density near the implant cause variability in recorded potentials across time. Minor meningeal encapsulation and gradual array movement may alter spike amplitudes, noise levels, and lead to loss of isolated units. These irreversible changes may nevertheless be compensable via algorithmic strategies.

Irreversible Non-Compensable Disruption: Severe meningeal encapsulation and array movement can progress to ejection of the device from the cortex, resulting in complete or near-complete signal loss which may disable the BMI.

### SECTION 2.3: Neuronal Degeneration

Device implantation results in a decrease in local neuronal density, particularly within 50 μm of the electrodes. As has been previously discussed, neuronal loss is attributable to a combination of traumatic damage during MEA insertion, the formation of a glial scar, and the neurotoxic and pro-inflammatory environment in tissue surrounding the MEA. During the acute post-implant phase (<4 weeks), local neuronal density may be dynamic. However, at chronic time points (>=12 weeks), both progressive neuronal loss and stable neuronal density can occur. At least some of this variability appears to be related to the specific method of device sterilization used pre-implantation. For instance, select sterilization techniques can result in a temporal decline in neuronal density between 2 and 16 weeks for certain device. Because microelectrodes may be sensitive to neurons within 140 μm of the recording site, local changes in neuronal viability are likely to substantially affect recordings.

Neurodegenerative or pathological states can occur near the implant site as early as 2-16 weeks post-implant. Also, Tau protein pathology, a characteristic form of neurodegeneration that is a consequence of neuroinflammation and microglia activation, can occur in axons surrounding implanted microelectrodes. Hyperphosphorylated tau causes this intracellular protein to misfold and clump into tangles inside neurons. In this regard, tau protein pathology may be associated with alterations in synaptic connectivity, abnormal spontaneous spiking activity, and changes in neuronal firing rates, which can contribute to destabilization of neural signals near the implant.

Other factors can contribute to neuronal degeneration and dysfunction. For instance, MEA implantation can be associated with loss of myelin near the electrode interface, a condition that impairs signal transduction of affected neurons. Furthermore, local dendritic loss can occur, which can affect synaptic processing and neuronal excitability. Observations of poor recording performance in the absence of both device material failure and severe neuronal loss suggest that some local neurons become impaired or silenced. Collectively, these degenerative and pathological states lead to neuronal signaling instabilities that affect both acute and chronic decoding performance.

Signal Disruptions Due to Neuronal Degeneration:

Irreversible Compensable Disruptions: Chronic neurodegeneration and neuronal dysfunction lead to inconsistent neuronal signaling and the potential for a gradual decline in the number of recorded single units. Although these conditions are irreversible, meaningful signal may still be recoverable through neural decoder feature optimization and other algorithmic strategies.

Irreversible Non-Compensable Disruption: In extreme cases of neurodegeneration or tauopathy, there is severe, irreversible signal loss not compensable through algorithmic strategies.

### SECTION 2.4: Inflammation and Infection

Apart from acute neuroinflammation associated with BBB breach, several other factors may cause or exacerbate the local neuroinflammatory response and recording signal disruption. For example, increased levels of residual endotoxins on neural implants after sterilization can cause greater microglial and macrophage activation, glial scarring, and neuronal loss at the implant site acutely after surgery. Activated glial cells near the electrode interface produce pro-inflammatory cytokines such as tumor necrosis factor alpha (TNF-α) and interleukin-1β (IL-1β) that can affect neuronal excitability and contribute to a neurotoxic environment. Neural tissue with increased expression of genes encoding for pro-inflammatory cytokines have been linked to reduced SNR in neural recordings. In addition, histological and gene expression analyses can cause heightened oxidative stress at the tissue-electrode interface. Reactive oxygen species (ROS), formed by inflammatory cells or during electrochemical reactions at the electrode surface, can deteriorate electrode materials, cause neuronal loss or degeneration, and potentiate neuroinflammation, resulting in MEA signal instabilities.

Stiff mechanical probes may propagate local neuroinflammatory cascades. Not only do mechanically stiff probes result in greater micromotion induced stresses, but they also decrease BBB integrity, increase glial scar density, increase neuronal loss, and increase levels of activated microglia and macrophages. Thus, the stiff silicon electrodes in such MEAs may exacerbate local neuroinflammation and contribute to signal deterioration.

Also, some BMI systems utilize transcutaneous connectors that have local skin sites that are prone to infection. Superficial infections may be treated with topical or oral antibiotics and may not affect MEA signal. However, deep infections spreading to bone that supports the connector could result in loosening of the screws leading to mechanical failure. In more severe cases, deep tissue infection could require surgical intervention and/or have other adverse health effects. In parallel, BMI users with a spinal cord injury or similar disability are at higher risk for systemic infections unrelated to the implant, e.g., urinary tract infections. For instance, peripheral inflammation may be linked to CNS modulation. Moreover, systemic infection may be associated in time with a decline in BMI decoder accuracy. Evidence that inflammatory responses are exaggerated in individuals with neurodegenerative disorders could mean that signal disruptions due to infection are more pronounced in certain clinical BMI populations than otherwise healthy subjects.

Signal Disruptions Due to Inflammation and Infection:

Transient Disruptions: Acute neuroinflammation or tissue edema after implantation may cause transient changes in firing rate that may resolve spontaneously when the underlying biological processes resolve.

Reparable Disruptions: Systemic infection is likely to cause altered neural signaling and recording instability that is reversible with systemic antibiotics.

Irreversible Compensable Disruptions: Chronic inflammation is associated with altered neuronal signaling, loss of recorded units, and a decrease in SNR that may be irreversible, but also potentially compensable with algorithmic strategies.

Irreversible Non-Compensable Disruption: Severe local deep tissue infections at the MEA implantation site may cause irreversible tissue changes, disruption of neural recording, and may require surgical intervention for device explantation.

### SECTION 2.5: Array Micromotion

Inconsistent neuronal firing rates and spike waveforms from the same MEA channel and subject may occur. For instance, it is possible that 61% of neurons may become unstable over 15 days. In humans, it is possible that 60% of units can become unstable after a single day, with firing rates and spike amplitudes varied for 84% and 74% of units, respectively, within a single recording session.

These instabilities likely arise from two sources: neurophysiological changes (discussed in the following section) and small fluctuations in spatial proximity between electrodes and neurons. In this regard, the synchronous shift in spike amplitudes across the array may be interpreted as evidence of micromotion causing signal variability. For instance, high acceleration head movements may be a contributing factor to array micromotion, as high acceleration head movements have been linked to abrupt changes in neuronal peak-to-peak voltages. However, different micromotion mechanisms may be at work in human studies, as severe signal instabilities have been identified in humans in the absence of rapid head movements. Additionally, abrupt electrode shifts are not consistent with the gradual loss of stable units observed in humans. Alternative explanations for micromotion include changes related to intracranial pressure, local vasculature, or biological processes occurring at the tissue-electrode interface.

Small shifts in array location may cause small changes in waveform amplitude (i.e., spike amplitude instability) that translate into significant impacts on apparent spike rate and BMI decoding performance. An illustrative example of spike detection error caused by a rapid baseline shift may result in 44% smaller spike amplitudes, which can be interpreted as a 50% drop in the unit's apparent firing rate because the spikes no longer met predefined amplitude criteria for the thresholding process. Offline spike resorting may reveal that the unit actually increased firing rate during this time.

Signal Disruptions Due to Array Micromotion:

Transient Disruption: Array micromotion may cause apparent changes in neuronal firing rates and spike amplitudes on the time scale of minutes to hours. Adaptive thresholding algorithms may help identify these situations and mitigate their effect on BMI performance.

### SECTION 2.6: Neurophysiological Changes

Acute changes in recordings may also result from neurophysiological changes in the recorded neuronal population. As an example, it is possible that approximately 85% of the observed instability in units may be due to variability in spike generation, e.g., as a result of cognitive and behavioral changes, neural plasticity, or other unknown physiological mechanisms as likely factors. Intracortical recording variabilities in somatosensory or motor areas may also be associated with the subject's emotional state, attentional state and arm posture. Neuroplastic changes from BMI practice or other neurorehabilitation methods may also alter neural representations across longer time scales. For instance, EEG recording is sensitive to acute variations in neural activity associated with medications, psychoactive substances such as caffeine, and physical and mental fatigue effects. As such, the extent these variations are represented at the level of MEA recording, e.g., in the hand/arm area of the motor cortex may be variable.

Other changes in firing rate may contain important information about context. For example, human motor cortex activity during control of a grasp neuroprosthetic may cause firing rates shifted in the presence of an object to be grasped. Therefore, in order to successfully perform BMI-controlled object interaction tasks, BMI decoding algorithms may account for the changes in neural firing attributed solely to the presence of the object as well as neurophysiologic changes associated with the user's intent to grasp it.

It is also possible that during reaching tasks, neural activity encodes not just arm kinematics, but also distinguishes between being in a state of rest versus holding a static reaching position. These changes in neural population tuning are important context-based signal disruptions that can interfere with prosthetic use and generate non-zero velocity predictions during rest if not recognized and properly handled. Thus, if used as assistive devices in everyday life, BMIs will be used in broader and potentially unpredictable circumstances, substantially contributing to context variability in neural representations.

Regardless of the underlying mechanisms, acute recording instabilities have the potential to negatively impact BMI decoding performance. For instance, firing rate instabilities may create a directional bias during cursor control strong enough to decrease target acquisition, e.g., from 100% to chance levels in as little as 30 minutes. To prevent such dramatic decreases, adaptive decoders may be utilized, which can update their parameters to account for instabilities. However, additional instability may result as the user continuously adapts to a regularly updating decoding model. An optimal balance between decoder adaptation and neural adaptation may improve BMI performance and robustness to disruptions. Ultimately, the deployment of portable intracortical BMIs may be necessary in determining the extent to which contextual and other physiological factors impact functional BMI performance.

Signal Disruptions Due to Neurophysiological Changes:

Transient Disruption: Changes in emotional, cognitive, environmental or physical states may cause acute variation in neuronal firing rates on the time scale of minutes to hours. Using adaptive machine learning decoders trained on substantial historical data may make BMIs robust to context-specific neural features.

Irreversible Compensable Disruption: Neuroplasticity associated with learning and practice may induce chronic, irreversible changes in neural representations that are compensable with algorithmic strategies.

### SECTION 3: Material Disruptions

Intracortical arrays are subject to ongoing biologic reactions that continually deteriorate components of the device. Explanted arrays exhibit evidence of these morphologic changes, which generally increase in severity with indwelling time. MEAs are susceptible to a variety of sources of transient and persistent noise whose effects can be exacerbated by material failures, e.g., damaged insulation or connector devices. These material disruptions act synergistically to degrade signal quality.

### SECTION 3.1: Pre-Implant Failure

Microelectrode array fabrication is an imperfect process, and defects have been noted even before the devices are exposed to the harsh in vivo environment. Material defects not only increase the risk of signal attenuation and corruption, but also prime the array for other sources of failure. For instance, the manufacturing inconsistency of planar silicon electrodes may partly explain variability in mechanical failure. For instance, pre-implantation, commercial parylene-C coated platinum/iridium (Pt/Ir) arrays may exhibit non-uniform insulation with cracking, as well as bent or cracked recording sites, which together may affect for instance, approximately 25% of the total electrodes. It is also possible that an array exhibits pre-implantation minor insulation delamination and irregularities. However, defective internal components may not be apparent, e.g., even when using imaging techniques to inspect an array prior to implant. However, according to aspects of the present invention, these pre-implant failures may be detected by outliers in impedance spectra.

By way of example, immediately following MEA implantation, electrodes can be identified as nonfunctional based upon the algorithms utilized herein. The precise etiology of this failure may be unknown. For instance, it is possible that some electrodes were damaged while handling the array just before implantation. Also, it is possible that the array experienced forces during cortical insertion. In other, more severe cases, manufacturing defects and improper sterilization techniques have caused complete array failure in NHPs. In conclusion, pre-implant disruptions are rare in clinical-grade devices; however, because they interfere with recordings indefinitely and can degrade signals by accelerating other failure mechanisms, they are still of high importance, and can be detected by an algorithm herein.

Signal Disruptions Due to Pre-Implant Failure:

Irreversible Compensable Disruption: A limited number of damaged or dysfunctional electrodes may irreversibly distort signals or cause loss of signal from individual channels. These disruptions may be compensable with algorithmic strategies to exclude or down-weight bad channels.

Irreversible Non-Compensable Disruption: Severe material defects during manufacturing have potential to cause irreversible, widespread signal loss that is not compensable algorithmically.

### SECTION 3.2: Insulation Deterioration

Over a dozen failure modes of microelectrode insulation may occur. Insulation damage increases effective surface area, allows for signal contamination by off-target cells, and provides a shunting path for signals which effectively reduces voltage amplitudes. Thus, insulation deterioration, irregular parylene-C and platinum (Pt) interfaces, delamination and cracking along the electrode shaft (with minor tissue invasion), and delamination near the silicon-coated wire bundle, may result in signal deterioration. Both cracking and delamination at the base of an array may result over time. However, more severe signal disruption effects of dielectric damage may occur for neural implants that utilize the same material for both the electrode and conductor because defect sites are equally capable of signal transmission.

MEA insulation is also susceptible to water absorption after implantation. Water absorption negatively affects dielectric properties and leads to attenuated signals, and electrical coupling to adjacent traces. Absorption also decreases impedance and increases phase. Also, water absorption of parylene-C reduces its adhesion strength and may contributes to dielectric delamination. Apart from tissue-electrode interface disruptions, complete array failure may be attributed to infiltration of water or other fluids at sites including external connectors.

In vivo cyclic voltammetry (CV) and electrochemical impedance spectroscopy can be used to identify current leakage pathway formation. Both the electrode yield and the number of recorded units are negatively correlated with cathodic charge storage capacity, suggesting that device integrity directly affects recording performance. Linear increases in the cathodic charge storage capacity over time suggest that deterioration such as cracking or delamination are ongoing processes throughout the indwelling period of the device. Additionally, irregularities in CV plots may be used to identify failures such as iridium oxide film delamination on stimulating electrodes. Stimulation parameters must be tightly controlled to prevent electrode degradation/degeneration, neural tissue damage, and undesired neural activation. Furthermore, disruptions to the reference electrode can result in high currents. Scan rates, particularly for MEAs with many electrodes, may impact the practicality of such measurements.

In contrast to CV, impedance measurements are easily and regularly obtained during clinical BMI recording sessions to assess recording and stimulating capabilities. Impedance characterization of devices has historically been reported at 1 kHz because it provides information about the exposed electrode area, and roughly matches the frequency of an action potential. For instance, 1 kHz impedance can correlate with recording metrics including array yield and the number of recorded units. For an example array with platinum recording electrodes, 1 kHz impedance less than 60 kΩ indicates shunting to ground. Active declines in impedance may signify ongoing insulation deterioration, formation of shunting pathways, and attenuation of recorded signals. Conversely, impedances of several MΩ indicate broken signal paths due to hardware failures or connection disruptions. Ultimately, information extracted from impedance measurements can be used to customize signal preprocessing and inform neural decoders to maintain long-term BMI performance despite signal disruptions caused by chronic material failures.

Signal Disruptions Due to Insulation Deterioration:

Irreversible Compensable Disruptions: Insulation failure can lead to irreversible signal disruptions including reduced signal amplitudes, off-target cell recording, and increases in crosstalk. Signal loss on select channels due to electrode shorting is also possible.

Irreversible Non-compensable Disruption: Catastrophic materials degradation or electrical shorting can result in irreversible, extensive and non-compensable signal loss. Electrochemical impedance spectroscopy can help identify material degradation and implant failures.

### SECTION 3.3: Electrode Degradation

Electrode materials in clinical intracortical BMIs are either platinum (Pt; for recording) or iridium oxide (IrOx; for stimulation). SEM imaging of explanted arrays generally show limited platinum degradation for recording devices implanted less than two years. At time scales approaching 1000 days, platinum corrosion, cracking, may occur, although some damage likely results from forces incurred during surgical explant. Nevertheless, the platinum-coated electrodes on arrays may be more stable than tungsten electrodes, which are known to corrode over shorter periods and produce toxic metal ions in the process.

Loss of platinum coating exposes the electrode's underlying silicon which dramatically increases impedance and decreases signal quality. Damage near the insulation/electrode boundary can provide another route for silicon exposure, resulting in signal change and corrosion that further undermines the metallic coating. Corrosion byproducts may also contribute to decreased signal quality through promotion of local inflammation. Optimal electrode materials can prolong high-quality signal acquisition, but over chronic periods, current devices are susceptible to electrode degradation that negatively impacts electrical properties.

Spatial topography of the electrodes (i.e., where the electrodes are positioned) provide additional evidence that material degradation can contribute to chronic impedance decline and signal deterioration. For example, electrodes nearest the wire bundle may experience a more rapid decay relative to other electrodes. Mechanical damage near the wire bundle interface may introduce material defects that accelerate other failure mechanisms including fluid infiltration and electrical bridging between channels. Furthermore, material degradation and increased channel crosstalk may be evidenced by a chronic increase in channel correlation.

Signal Disruptions Due to Electrode Degradation

Irreversible Compensable Disruptions: Damaged electrodes may cause irreversible distortion or loss of signal that may be compensable through algorithmic strategies.

Irreversible Non-compensable Disruption: Catastrophically damaged electrodes can result in irreversible, extensive and non-compensable signal loss and array failure.

### SECTION 3.4: Signal Noise

Intracortical recording systems are susceptible to both biotic and abiotic sources of noise. Major sources of biotic noise include ionic activity from “background” neurons firing, nearby muscle activity, and motion artifact. Microelectrodes are sensitive to neurons within −140 μm of the recording site. Thus, signals acquired from a single electrode could be influenced by dozens or hundreds of neurons depending on implant location, local neuronal viability, and degree of tissue encapsulation. Activity from distant neurons may be difficult to effectively isolate and therefore has traditionally been considered signal noise. As such, changes in neuronal density and firing rates contribute to non-stationary biological noise. Also, abrupt motions or nearby muscle activity can produce artifacts in recorded signals that are common across all electrodes. In this regard, common noise may appear with similar characteristics to neural activity, demonstrating that traditional noise rejection methods such as differential referencing can be inadequate. Similar deficiencies in eliminating motion artifact may also apply to common averaging referencing as well. Moreover, the degree of local tissue resistivity from device encapsulation may correlate with thermal noise.

Abiotic noise arises from BMI hardware and environmental interference. Contributions from recording systems include electrode-electrolyte interface noise and electronic thermal and flicker noise. High-density arrays may be particularly susceptible to cross-talk, which can attenuate recorded potentials and influence signals in adjacent electrodes. Systems that employ high-density arrays and/or wireless data transmission may also have to contend with hardware constraints that limit data bandwidth. Consequently, these systems may need to employ strategies such as lossy data compression that degrade signal quality.

Environmental noise primarily presents as electromagnetic interference (especially at 60 Hz), but other artifacts such as electrostatic discharge may occasionally disrupt recording. BMI systems that incorporate functional electrical stimulation (FES) to restore hand or arm function, or intracortical microstimulation for somatosensory feedback, are particularly susceptible to extreme levels of electrical artifact. Large voltage transients during such stimulation periods decrease neural decoding performance in the absence of compensatory algorithms. Even in cases where the sources of noise are small in magnitude compared to recorded action potentials, the cumulative effect ultimately acts to lower SNR and decrease neural decoding performance. As BMI systems grow more complex to support multiple and more diverse end-effectors, and are used in new environments, recorded noise levels will continue to become more variable.

In some embodiments, signal artifacts are categorized under signal noise for readability and flow. However, in some embodiments, a distinction can be drawn between signal artifacts and signal noise.

Signal Disruptions Due to Signal Noise:

Transient Disruptions: Sources of noise, including electrostatic discharge, stimulation transients, and motion artifact commonly cause transient signal artifacts. Contextual environmental noise may also variably influence recordings. These sources of noise can frequently be cleaned from the signal using algorithmic methods.

Irreversible Compensable Disruption: Background neural activity can introduce irreversible signal noise that cannot be robustly isolated but can be mitigated through careful neural feature selection and algorithmic strategies.

Irreversible Non-Compensable Disruption: Recording and effector devices are sources of irreversible, inherent noise that are not amenable to algorithmic compensation.

### SECTION 4: Mechanical Disruptions

Neural recording systems are susceptible to mechanical interferences at both micro- and macroscopic levels. At the microscopic level, micromotion of the array and mechanical agitation of surrounding tissue are the dominating disruptive modes. However, the mismatch of mechanical properties between the cortex and implants generally manifest as biological disruptions through neuroinflammation, and as such, are covered in previous sections. At the macroscopic level, hardware failures such as faulty connections or physical trauma could rapidly change recordings or cause permanent dysfunction.

### SECTION 4.1: Traumatic Damage

Intracortical MEAs in clinical recording systems currently require a transcutaneous, bone-anchored port to transmit data. Cables that connect to the port have a tall rigid base that can act as a lever to produce large, destructive forces on the connector and skull. Accidental trauma to the connector or forces applied by the cable could result in unrecoverable damage to the system or user. Acute traumatic damage to intracortical MEA systems is the most common failure mode for NHPs. The mechanical reliability of skull-mounted connectors has prompted the design of accessory hardware to enhance connector stability. MEA connectors are also susceptible to localized physical damage that can affect recording channels. For instance, the surface of a skull-mounted CerePort connector may have an exposed gold connector pin for each electrode in the array. These pins are prone to irreparable damage caused by contact with headstage guide pins and other objects. Utilizing fully implanted systems would reduce the opportunity for external mechanical damage, but trade off power consumption, device size, and data transmission bandwidth to successfully translate fully implantable technology for human BMI applications. Furthermore, implanted hardware in BMI systems are also at risk for traumatic damage. Components such as electrode traces may suffer mechanical damage, particularly at high-strain areas caused by mechanical property mismatches or device geometry. Lastly, cases of head trauma may disrupt recording by damaging neurons and microvasculature near an implant. Traumatic brain injuries can initiate neuroinflammation, alter intracranial pressure, contribute to chronic neurodegeneration, and significantly alter the landscape of recordable neurons.

Signal Disruptions due to Traumatic Damage:

Irreversible Compensable Disruption: Irreversible signal distortion may occur due to minor damage of irreplaceable hardware components, such as external gold electrode pins. Distortions may be compensable with algorithmic approaches.

Irreversible Non-Compensable Disruptions: Traumatic damage to the skull mounted connector or internal wire bundle can cause irreversible, non-compensable disruption and inability to record signals. Head trauma may also result in irreversible neural dysfunction depending on the severity, and it is unclear how algorithmic techniques could improve signal quality after these events.

### SECTION 4.2: Connection Failures

After neural activity is acquired through the microelectrodes on the array, the signals are transferred through a series of cables and connectors, each of which has potential to fail independently. For example, the filament interface between a CerePort and headstage can accumulate debris that prevents proper interfacing and corrupts signals. Analog headstages are particularly susceptible to noise and can require complicated amplifier connectors to support high numbers of recording channels. Improvements in connection reliability and signal noise can be achieved with headstage hardware that digitizes neural signals near the recording site. These digital headstages are also more compact, and less obtrusive, which may enhance their integration in portable BMI systems.

By way of illustration, impurities between a connector and head stage can cause poor contact, which may result in, e.g., a twofold increase in noise and the disappearance of spikes. Here, it is possible that a contact may open and close dynamically depending on movement. Cleaning the connectors may enable the recovery of malfunctioning channels. For example, if training data for a decoder were collected using a compromised cable, hardware maintenance may alter recorded signals, and ultimately decrease BMI performance. Connection disruptions can also cause high variability in electrode impedance measurements and impair recording consistency. Although many connection disruptions can be remedied by a technician or a replacement part, further damage is possible during system repair. For instance, improper cleaning of CerePort contact pads may cause recording system failures.

Faulty connections are often overlooked as significant failure modes for BMIs, but as these systems become portable and are used without technician oversight, the severity and likelihood of connection disruptions increases. Any system dependent on connection hardware is at risk for faulty connections, cable damage, or hardware malfunction that could interfere with signal transmission. Also, connection disruptions are possible every time the user connects to and disconnects from the system. Given the potential for signal disruptions to be masked during signal processing, e.g., utilizing normalization methods or insensitive feature extraction, aspects herein may establish careful data checks as standard operating procedure for device use. Furthermore, especially for clinical BMI systems with stimulating microelectrodes, safety procedures require identification of connection disruptions to appropriately disable electrodes and prevent irreversible damage from exposure, e.g., to high voltages.

Signal Disruptions due to Connection Failures:

Transient Disruptions: Unstable connections may cause temporary loss or gain of viable recording channels. Hardware maintenance may promote the recovery of viable channels.

Reparable Disruptions: Faulty external cables or connections can cause persistent channel crosstalk, interference, or signal loss. These disruptions can be corrected through repair or exchange of the faulty hardware.

### SECTION 5: Discussion

As noted herein, common MEA signal disruptions of biological, material or mechanical etiologies can further be classified according to their duration and amenability to repair or compensation. This shift in focus from the cause of disruption to characteristic effects on signal and BMI performance provides opportunities to consider how each type of disruption is best identified and what interventions might enable recovery of high-quality signal. Intracortical MEAs are subject to a dynamic in vivo environment which, if not accounted for, will render static neural decoders ineffective over relatively short periods. The potential for signal disruptions will further increase as BMI systems transition from being experimental devices used in controlled laboratory settings to portable devices used in the multiple unpredictable environments of daily life. Neural recordings will be subject to unique and varied sources of environmental noise, while hardware components will be at risk of interference, physical damage, and unanticipated challenges in use cases. Additionally, the cognitive state of the user and the context in which the device is operated will be highly variable, affecting neural responses in unpredictable ways. However, aspects of the present invention utilize machine learning and statistical methods to mitigate the diverse range of signal disruptions encountered by BMIs.

By way of example, in vivo diagnostics and algorithmic approaches may be utilized to detect ongoing signal disruptions. Moreover, strategies are utilized to combat transient, reparable, and irreversible compensable disruptions.

A novel, automated approach for dealing with corrupted channels includes (1) automatically identifying problematic channels adapting using established statistical process control (SPC) techniques, (2) inserting a masking layer in neural network decoder architectures to remove the problematic channels without retraining from scratch, and (3) unsupervised updating to reassign the weights of the remaining channels without requiring the user to explicitly recalibrate. As discussed herein, using SPC, key channel health metrics like impedance and channel correlations are monitored over time, yielding baselines and tolerance bounds for normal operating behavior. Channels within the tolerance bounds pass through the model unaltered. If any channel metrics exceed the tolerance bounds, the identified channels are determined disrupted and then removed in the channel masking layer so they cannot influence subsequent decoding layers. Importantly, the channel layer removes channels without changing an underlying model architecture, which enables methods such as transfer learning and fine-tuning to adapt the decoder in a computationally efficient manner. Unsupervised updating can then continually improve the model without placing any additional burden on the user. SPC methods are completely independent of the neural decoder and can be applied wherever there is sufficient historical data to establish a baseline and assess variability. The masking and unsupervised updating approaches are flexible and can be used with any existing neural network architecture.

### SECTION 5.1: Disruption Detection Methods

Identifying ongoing disruptions are associated with targeted algorithmic countermeasures. In this regard, in vivo impedance spectroscopy is utilized as a diagnostic tool, to reveal unique impedance signatures for varying degrees of microelectrode tissue encapsulation. Impedance spectroscopy, in combination with equivalent circuit modeling, provides insight on abiotic failure modes such as insulation deterioration, wire breakage, and electrode tip degradation/degeneration. Cyclic voltammetry may also be used to identifying the formation of current leakage pathways. Notably, cathodic charge storage capacity may actually increase with implant time and negatively correlate with electrode yield and the total number of units recorded. The techniques herein may not be practical with current MEAs in humans.

In practice, disruptions can coincide and have overlapping effects that confound diagnostic metrics. For instance, tissue encapsulation of MEA electrodes raises impedance, while insulation deterioration creates shunting paths that lower impedance. Though some disruptions may occur over characteristic time periods (e.g. insulation water absorption and tissue encapsulation following device implantation), compounding effects make it challenging to determine underlying failures precisely. Nevertheless, relationships between impedance and common device failures can leverage in vivo diagnostic techniques to predict recording channels that attenuate signals, or channels that are likely to worsen with time. In some embodiments, these predictions are utilized when selecting neural features such as channel-wise spike amplitude thresholds. It is also feasible that these predictions could inform decoding models to maintain performance over prolonged periods.

Automated real-time monitoring of signal quality may be a component of fielded BMI systems. In this regard, according to aspects herein, statistical process control (SPC) is utilized for signal quality monitoring. SPC can be applied in a BMI context by monitoring signal metrics such as impedance, channel correlations, and SNR, and checking for deviations from baseline as well as outlier channels that may indicate hardware failures. For example, insulation degradation can lead to electrical shunting, which may be detected by abnormally high correlation between adjacent channels. Monitoring impedance is useful for detecting several disruptions, ranging from irreversible electrical shorting due to severe materials degradation/degeneration, to reparable disruptions such as a loose headstage connector.

In an example embodiment, following the automated identification of abnormalities by SPC algorithms, technicians are alerted and/or decoders are updated to compensate for channels exhibiting abnormal behavior.

The disruption-identification process comprises three general steps: (1) transformation of the raw neural data into array-level metrics appropriate for SPC, (2) flagging of days with out-of-control signals via SPC, and (3) identification of individual problematic channels using an outlier test (e.g., Grubb's outlier test, Dixon's Q test, Chauvenet's test, etc.) when the array-metrics are deemed out-of-control.

Transformation of raw neural data into array-level metrics: primary metrics based on both channel impedances and voltage recordings can be used to detect signal disruptions, as discussed above. The SPC approach could be used to monitor any number of BCI metrics, but impedance, voltage data of electrostatic discharge artifacts (Vrange), and channel correlations (minimum and maximum) should be enough to identify disruptions in most systems. Vrange was calculated as the difference between the maximum and minimum voltages recorded for each channel after a 250 Hz fourth-order high-pass Butterworth filter had been applied. This calculation differs from the standard calculation of peak-to-peak voltage, which aims to measure the quality of the action potential by taking the difference between the maximum and minimum voltages at threshold crossings rather than over the whole signal as is done for the Vrange calculation. This metric is expected to detect connector disruptions and sources of abnormal artifacts such as floating channels. For the channel correlations, the voltage recordings were also used to calculate 96×96 matrix of pairwise correlations between channel voltages, which were then used to determine minimum and maximum correlation values.

Flagging of days with out-of-control signals: control charts were produced for each of the four metrics above to identify days with abnormal signal behavior. For the development of this method, the selection of the control limits was guided by identification of known disruptions in the data. We set control limits at 2.66 standard deviations away from the mean for x̅-charts and three standard deviations away from the mean for S-charts. The Type I error rate for each control chart, the probability that a given datapoint appears out-of-control when the result is in fact due to random variation, is approximately 0.8% for the two-sided X̅ charts (0.4% for the maximum absolute correlation x̅-chart, where only the upper control limit is considered) and approximately 0.3% for the S-charts. These values can be tuned to balance sensitivity and specificity appropriately.

Classical SPC methodology assumes that in the absence of a disruption, data follow a Gaussian distribution with a constant mean and variance. However, these assumptions were not initially met for the metrics produced. For example, channel impedances were found to decay with decreasing variance over time. Impedances were instead found to approximately follow an exponential decay model with stable variance over the log of time. Similar properties were observed for Vrange and the correlation-based metrics. Therefore, exponential decay models were fit to impedance and Vrange metrics. Correlations between channels increased slowly over time, and thus logarithmic growth models were fit to the correlation-based metrics. Model residuals were used to construct control charts. To simplify interpretation, all control charts were standardized. That is, the control limits are kept constant for the duration of the study, and the datapoints are scaled accordingly.

Identification of individual problematic channels using the Grubbs test when the array-metrics exceed the control limits: the SPC approach described herein flags problematic days based on array-level metrics. Thus, once a day has been flagged, the problematic channels still needed to be identified. To identify which channels were disrupted, the Grubbs test for outliers was performed across all channels and for each control metric on each day flagged by the control charts. A significance level of 0.01 was used. The Grubbs test assesses whether the largest absolute deviation from a sample mean is significantly higher or lower than expected for each channel based on the assumption of normally distributed data. For each channel, if the flagged session with the largest deviation from the mean for a given channel is identified as an outlier, the session is removed from the dataset and the test is performed again on the next most deviant flagged session. This process is repeated until no new outliers are found. Only channels that both were classified as outliers by Grubb's test on two or more consecutive days and occurred on sessions flagged on the control charts are deemed “corrupted” and would be subjected to masking. The same channels must be identified on both days to be considered outliers. The SPC algorithm is tuned to detect the types of disruptions that the current system was susceptible to, and thus which might be expected in similar systems by selecting parameters based on identifying known disruptions and based on the amount of error that was deemed acceptable in identifying false disruptions or missing true disruptions. These parameters include the limits for each type of control chart, the number of consecutive days of out-of-control observations required before a metric would be flagged, and the significance level of the Grubbs test. The transformations applied were selected because they improved conformity of the data to assumptions of normality and constant variance. The above settings and transformations can be easily customized to comply with the needs and allowable risks of future systems while maintaining the same general SPC methodology.

In some embodiments, BMIs leverage statistical approaches to detect transient disruptions such as array micromotion that cause rapid, unexpected changes in firing rates and spike amplitudes. Similar to irreversible and reparable disruptions, in some embodiments, early detection of transient disruptions initiate neural decoder adjustments to mitigate the effects on BMI performance. Furthermore, dramatic drops in BMI performance in the absence of statistical outliers may indicate deficiencies in signal processing and decoding. Ultimately, these signal monitoring approaches help ensure BMIs are functioning properly for extended periods of time and quickly identify problems that may require intervention.

### SECTION 5.2: Algorithmic Strategies for Transient Disruptions

BMI operation may be influenced by recording instabilities including array micromotion and transient noise, as well as physiological factors such as cognitive or contextual changes that affect intrinsic spike generation (e.g., sections 2.5, 2.6). Even in well-controlled environments, BMI performance may continually degrade because of gradual changes in spike rates and signal amplitudes from unstable units. In this regard, BMI performance may be improved by reducing the effects of these transient disruptions and eliminating the need for regular system recalibration. In this regard, neural feature engineering, neural decoder training strategies, adaptive neural decoding methods, and signal filters and referencing techniques can assist in mitigating the effects of transient disruptions. Although each strategy is discussed separately below, in practice, these techniques can be combined in any manner to further improve robustness.

An approach to prevent declining accuracies due to transient disruptions is to use neural features that are designed to be robust against these disruptions. Historical recordings and extracellular waveform characteristics can be leveraged to identify stable units for decoder training. However, this approach restricts bandwidth by excluding potentially useful information from recordings. An alternative solution is to use neural decoding features that are minimally susceptible to recording instabilities. Threshold crossings are vulnerable to amplitude shifts in neural recordings, whereas features based on spectral power may be more robust. BMIs may also leverage neural manifolds, low dimensional projections that capture much of the variance in neural population activity, to combat transient disruptions. In an embodiment herein, neural activity is stabilized by aligning manifolds across time. This approach can counteract recording disruptions including changes in baseline firing rate and neural tuning, as well as loss of recorded units.

Another approach to build robust decoders is data curation and training of the decoder parameters. In a laboratory context, a decrease in BMI performance due to task-related neural modulation can be alleviated by training neural decoders under similar conditions to the use case. However, it is impractical to train take-home systems under every possible use-case of the BMI. Instead, deliberate neural decoder training strategies and data augmentation make BMIs resistant to transient disruptions. Using large amounts of historical data to train neural decoders increases the likelihood that a given model will be exposed to a variety of signal disruptions. By training with datasets containing disruptions, machine learning models become more robust to similar disruptions that occur in the future. Training data can also be artificially enhanced by simulating perturbations in neural decoding features that are representative of transient disruptions. These approaches may improve decoder robustness not only to recording instabilities such as array micromotion, but also to neural variability across cognitive and behavioral contexts.

Another strategy is to use adaptive decoding models that combat signal instabilities through recurring parameter updates. Adaptive decoders can outperform their counterparts with fixed parameters because they account for ongoing disruptions in neural recordings. For certain BMI applications such as virtual typing, user intention can be inferred retrospectively and used to facilitate updates. Recalibration methods may also use recent neuronal activity and decoder predictions obtained during BMI use to update the model, circumventing the requirement for explicit training labels. These self-recalibrating procedures eliminate the need for daily retraining, and therefore minimize BMI setup time. Despite the performance of adaptive decoding algorithms over chronic periods, in rare cases, disruptions can still cause neural decoding accuracy to vary, e.g., by up to 20%. These adaptive machine learning methods can be enhanced with the decoder training strategies and neural features previously discussed.

Also, some transient disruptions, including electrical artifacts, may be mitigated by selection of referencing techniques and data filters. Common average referencing aims to remove noise and artifacts common to all electrodes by re-referencing recordings to the average potential across channels. In clinical BMI systems, a subset of electrodes with the lowest root-mean-square values are used to calculate this reference. Though common average referencing can improve SNR, it can be inadequate for certain artifact removal applications because it assumes noise is similar across all electrodes. For removing FES artifacts, a channel-specific referencing method can be utilized. In this regard, a channel-specific referencing method can outperform common average referencing and artifact blanking. In addition, neural information can be recovered during FES stimulation periods, even when the artifact is orders of magnitude larger. Signal quality may be further enhanced by optimizing data filters. For instance, high-order filters that produce oscillatory artifacts in recordings can decrease BMI decoding accuracy. Furthermore, non-causal bandpass filters can yield greater spike amplitudes and improved decoding accuracy compared to their equivalent causal filters. Synergistic approaches that combine multiple signal processing and decoding methods may thus be utilized to effectively suppress transient signal disruptions.

### SECTION 5.3: Algorithmic Strategies for Reparable Disruptions

Ultimately, system set up may be performed by caregivers instead of trained technicians, increasing the likelihood of faulty hardware connections or errors during neural decoder training. Poor connection to a percutaneous pedestal causes recording inconsistencies, reduces total available neural information, and increases the risk of reversible and/or irreversible damage to stimulating microelectrodes and surrounding tissue. In order to support human-machine interface use outside the lab and without a technician, the automated algorithms herein can be utilized to quickly identify such malfunctions (which would otherwise go undetected for substantial periods without technicians regularly checking signal quality) and to mitigate effects of the malfunctions before the human-machine interface can be repaired.

### SECTION 5.4: Algorithmic Strategies for Irreversible Compensable Disruptions

Irreversible disruptions frequently affect intracortical BMIs because the neural interface and much of the associated hardware is inaccessible without surgical intervention. Consequently, biological responses or damage to the recording device may cause permanent changes in acquired signals. Though in rare cases irreversible disruptions can result in catastrophic signal loss, many of these disruptions can be compensated for algorithmically.

Most irreversible compensable disruptions contribute to chronic attenuation or loss of recording channels. These effects can devastate BMIs with vulnerable decoding methods. For instance, the loss of just three neurons from a stable neural ensemble could decrease online BMI accuracy by, e.g., up to 50 percent. However, aspects herein mitigate the effects of irreversible disruptions by leveraging data augmentation techniques during decoder training. Also, artificially perturbing firing rates during decoder training may make the decoder more robust to this disruption.

Similar to compensatory strategies for transient disruptions, adaptive decoding methods that down-weight the influence of permanently damaged channels can maintain decoding accuracy in the face of irreversible disruptions. However, this strategy will become less effective with the accumulation of irreversible failures over time. If there is insufficient information in remaining channels to maintain BMI performance, algorithmic compensation becomes increasingly difficult. However, it is possible that neural dynamics under the same motor behaviors may be reliable across time, regardless of recording quality. As such, neural population dynamics inferred from historical recordings with high neuron counts can be leveraged to rescue neural decoding performance after severe electrode loss, thus extending functional BMI lifetime.

Gradual declines in signal quality due to loss of units or material degradation/degeneration may also be counteracted with targeted neural decoding features. As signals attenuate with time, and it becomes difficult to attribute electrical potentials to particular neurons, BMIs may benefit from features that salvage information from subthreshold neural activity. As an example, mean wavelet power may utilize weak or distant spiking information that is sometimes considered biological noise.

In some embodiments, mitigating algorithmically covers everything in section 5.4, e.g., including optimizing decoders, data augmentation, machine learning strategies, combinations thereof, etc. In other embodiments, the term mitigating algorithmically covers a subset of that covered in section 5.4, in any combination thereof.

### SECTION 6: Conclusion

The algorithmic strategies herein can be used alone or in any combination, in concert to mitigate the vast range of potential disruptions that intracortical BMIs face. As the diversity of BMI effectors expands from computer cursors to sophisticated devices that interact with the environment, the consequences of inaccurate predictions increase. Misspelling a word is inconvenient, but the inability to accurately control a robotic arm may pose a danger to the user and others around them. Therefore, it will be even more critical to ensure that BMIs are resilient to recording disruptions. As such, aspects herein categorize many of the common signal disruptions in order to guide targeted algorithmic solutions. Creating systems that can detect and compensate for these disruptions facilitates the translation of BMIs from the laboratory setting to a portable assistive device.

### SECTION 7: Example Embodiment of Sensor Compensation Process

### 7.1 Damaged Channels Identified by the SPC Process

Statistical process control methods that have been adapted for use with neural data can reliably identify problematic channels. Three examples of identifying damage are shown below.

Two electrodes shorted together (e.g., by a scrap electrode pad that had fallen loose) may be detected by on the X̅-chart for maximum absolute pairwise channel correlation of the SPC process for seven out of the ten sessions between days 871 and 906 after implantation, when abnormally high and out-of-control correlations were observed for these two channels. The electrical short effectively duplicated the signals across two channels and therefore the subtle effect was only apparent by observing the abnormally high correlation between the two channels. The correlation of these channels returned to being in-control after physical repairs of the system were made.

As another example, a bent amplifier pin can be detected using the impedance s-chart. Pins may become bent due to repeated connections and disconnections. An indication of an impedance 4-15 standard deviations about the mean on the impedance S-chart can show such a bent pin. When repaired, the impedance will return to normal/expected values.

A further example includes identifying electrically floating pins due to material degradation of the MEA, the percutaneous pedestal, or both by looking at noise recorded instead of actual neural activity on those channels/pins. The noise manifests in the data as abnormally low correlations with surrounding non-floating channels and high correlations amongst the floating channels. Consequently, both the S-chart for Vrange and the X̅-chart for minimum average channel correlations will show out-of-control observations when floating.

The modified SPC approach presented here was able to successfully identify three known occurrences of damage to the MEA channels, and misclassified normal neural signals as disrupted less than 20% of the time. Thus, this new manner of identification and classification provides a benefit over previous ways of identifying and classifying.

### 7.2 Model Performance with Damage to the MEA

A first method of adjusting to damaged channels includes an unsupervised neural network (uNN) trained using all channel inputs over the training period and given unsupervised updates for each test session was tested with no explicit adjustments made to accommodate the damaged channels as a control condition (uNN-NOMASK).

A second method of adjusting to damaged channels includes a uNN framework trained using all channel inputs over a training period and given unsupervised updates on each test session with the masking layer inserted immediately prior to the decoder architecture to zero out the corrupted channels (uNN-MASK). The decoder architecture was identical to that of the uNN-NOMASK except for the addition of the masking layer.

A third method of adjusting to damaged channels includes a uNN model retrained from scratch from the beginning of the training period and given unsupervised updates on each test session with input from the damaged channels removed from the array (uNN-RETRAIN). The decoder architecture was identical to that of the uNN-NOMASK except the initial layer was modified to accommodate the reduced number of channels. For example, in the case where the top 10 most important channels were artificially corrupted, the model would take an 86×9 dimensional array as its input instead of the regular 96×9 array. This method is the most intensive of those tested in terms of data storage requirements and retraining time.

A fourth method of adjusting to damaged channels includes a support vector machine (SVM) trained from scratch using only labeled data from the first block of the day of the test and with input from the damaged channels removed from the array (SVM-REMOVE). This model is trained as an additional baseline.

Comparing the uNN-NOMASK model to the SVM-REMOVE model, the uNN decoders (which were all equivalent when no corruption was introduced) achieved superior performance to the SVM in the baseline scenario when no channels were disrupted. When no damage was introduced to any of the channel results, the uNN decoders achieved a mean accuracy of 89.87±6.97% (mean±standard deviation) and a success rate of 90.97±15.61% over the test period. The SVM-REMOVE achieved a lower mean accuracy of 83.20±6.23% and a success rate of 77.90±6.23%.

The low relative performance of the SVM-REMOVE continued through the disruption of the 10 most important channels, such that it was the poorest-performing model in these cases. However, the performance of the uNN-NOMASK decreases drastically after 15 or more channels were affected, and falls far below the performance of the other models. The performance of the uNN-NOMASK was falls near or below chance levels when 15 or more of the most important channels are affected, and so can provide little to no benefit to the user in these cases.

The uNN-NOMASK required an average of 63 batches for the daily unsupervised updating procedure regardless of the amount of simulated damage introduced. A total of 31 blocks of data storage were used, 11 of which were used for the daily update procedure and 20 for the validation dataset. The daily-retrained SVM would only require one block of data stored at a time and can be trained in fraction of the time of a deep learning model, but importantly, would require the user to actively spend time each day collecting labeled data which is not required for the uNN models.

These results demonstrate that a uNN approach is superior to the SVM approach in most use cases when no or minimal array damage is present, both in terms of accuracy and in terms of the computational and time burdens on the user. When greater amounts of damage are present in the array, additional means of addressing the damage are necessary for the uNN to be viable.

Both uNN models with that explicitly handle the corrupted channels maintained high performance through a considerable amount of corruption introduced to the neural signal. Unlike for the uNN-NOMASK and SVM-REMOVE, mean accuracies remained above 85% and mean success rates remained above 80% for both the uNN-RETRAIN and the uNN-MASK for corruption of up to the 20 most important channels.

Minimal additional data is required to adjust the uNN with the channel masking layer in place compared to the uNN with no masking, as the uNN-MASK only utilizes data used in the daily unsupervised updating procedure. The computational time for the uNN-MASK is simply that needed for the unsupervised updates. The average number of batches needed to update the uNN-MASK ranges between approximately one (for one channel affected) and three (for 50 channels affected) times the number needed to update the uNN-NOMASK is implemented. This increase in computation time over the uNN-NOMASK is due to additional epochs being needed to reassign weights when more channels are masked.

For cases in which damage was introduced, the uNN-RETRAIN attained accuracies and successes that were superior to or statistically not different from all other models. Similar to the uNN-MASK, accuracies for the uNN-RETRAIN were significantly different from the baseline model with no corruption when 10 channels were disrupted and success rates for the uNN-RETRAIN were not statistically different from the baseline until or more channels were affected.

However, retraining the uNN from scratch only yields statistically better accuracy and success compared to masking in greater instances of damage, when 30 or 50 channels are affected. The maximum average benefits in accuracy and success obtained by retraining from scratch over masking were only 4.69±3.96% and 24.55±17.03%, respectively, which were each attained when 50 channels were disrupted. Performance was relatively poor for both the uNN-RETRAIN and uNN-MASK when the 50 most important channels were disrupted, when both attained accuracies below 75% and success rates below 40%.

Retraining the model from scratch with the bad channels excluded requires much more processing time and can take between approximately four (for 50 channels removed) and ten (for one channel removed) times longer on average than simply performing the unsupervised update after masking. In contrast to the 31 blocks of data used to implement the uNN-MASK, a total of 81 blocks of data were used to retrain the model from scratch. This included all sixty blocks of data of the labeled data from the training period would be needed in addition to the twenty blocks of validation data. As the test period continued, 11 blocks of data used for daily updates were also added to the data stored.

Unsupervised updating allows the algorithm to adapt to gradual changes in the neural signal over time. However, the updating process is also critical for the success of the channel masking procedure. The maximum masking benefit a model that did not receive unsupervised updates was only 15.2±5.00% in accuracy and 44.61±17.11% in success, which occurred when 15 corrupted channels were masked. In contrast, when unsupervised updating was performed the accuracy and success increase by 23.57±5.68% and 58.19±17.78%, respectively, for 15 channels affected. The model that masked corrupted channels also performed statistically significantly worse than the model that was retrained from scratch when updates were not performed after only 10 channels were affected (accuracy: 2.17±2.44%, t(8639)=−3.78, p=0.0006; success: 4.86±9.44%, t(8639)=−3.52, p=0.0017. The unsupervised updating procedure is necessary to readjust the weights after the most important channels have been omitted, and thus those which the decoder had been most reliant on for information may be dropped during updating.

Traditional statistical process control techniques were adapted to identify several outlying signals that may correspond to damage to the neural sensor itself or to downstream components. The benefits of this automated damage-identification algorithm are twofold. First, the SPC procedure can alert the user or technician when a potential issue occurs, expediting repairs and triggering algorithmic adjustments. This benefit exists independently of how well the decoder adapts to the damaged channels. Second, identifying problematic channels allows them to be explicitly handled by the decoding algorithm.

As discussed above, the overall success rate of the uNN-MASK with damage is only a few percentage points lower than in the case of no damage when up to fifteen of the most important channels in the sensor are masked. Even when 20 of the most important channels are masked, the uNN-MASK success rates remain above 80%. This is a lower success rate compared to the model's typical performance, but would still satisfy approximately three quarters of potential BCI users with spinal cord injury surveyed. In contrast, the uNN-NOMASK successfully responds to cues less than a third of the time when 15 channels are corrupted. When damage was simulated in 20 channels, the uNN-NOMASK was almost completely unusable.

Further, the benefit obtained from channel masking is immediate, such that performance of the uNN-MASK is near the performance of the uNN-RETRAIN on the first day after the channel masking is implemented. Benefit from the masking layer is the most critical within the period immediately after neural signal disruptions are discovered because many cases of damage would ideally be repaired within the first few weeks after detection. Even when labeled data is used to update the model in a supervised fashion for the first several days after damage is detected, performance is higher with channel masking turned on versus channel masking left off. This further highlights the benefits of explicitly masking damaged channels, especially considering that benefit obtained from channel masking is the most crucial for the first few days after damage.

By leveraging previously learned weights that already encode much of the necessary information for decoding, the MEA can efficiently compensate for disruptions and damage without compromising performance.

Some robustness to changes in the neural signal was also incorporated during the training stage through the use of dropout, which simulates loss of a random set of connection weights in each layer in order to regularize the model and prevent over-reliance on a given set of connections, and mixup, which augments the training dataset by generating linear combinations of training examples. However, the poor performance of the uNN-NOMASK for more than 10 channels affected shows that these training strategies are not sufficient to maintain high performance of the BCI.

Some robustness to changes in the neural signal was also incorporated during the training stage through the use of dropout, which simulates loss of a random set of connection weights in each layer in order to regularize the model and prevent over-reliance on a given set of connections, and mix up, which augments the training dataset by generating linear combinations of training examples. However, the poor performance of the uNN-NOMASK for more than 10 channels affected shows that these training strategies may not be sufficient to maintain high performance of the BCI.

These results also add to the evidence that the unsupervised updating approach for neural networks described herein has significant benefits for sustaining the robust performance requirements of BCI users while maintaining a light computational footprint that is compatible with the low-power devices that will deploy these algorithms. The benefit attained by masking increased substantially when masking was accompanied by unsupervised updates to readjust model weights. Furthermore, after damage is repaired, the previously masked channels will need to be reintroduced back into the model. The reintroduction is as simple as changing the weights in the identity layer for the relevant channels from 0 to 1. However, the decoder will have adjusted its weights to ignore input from the previously masked channels. Therefore, the unsupervised updating procedure will again be critical for readjusting the weights to reincorporate the repaired channels.

The conventional approach of addressing disrupted array signals by retraining the decoder from scratch with the damaged channels removed requires significant time from the participant to collect new training data followed by computation time to retrain the decoder model before the decoder is usable. Furthermore, if the decoder uses historical data, that data needs to be stored and accessible for retraining the model. The approach described herein substantially lowers both the computational and storage burden compared to entirely retraining the model. The time and data storage requirements for the SPC approach to detect disrupted electrode signals are negligible. This scheme only requires that up to two values (one corresponding to each the X̅-charts and potentially the S-charts) be stored for each of the four metrics per day. The entire procedure only requires a small amount of rest data collected each day and only a few simple calculations.

Adjustments to the model to accommodate the masked channels occur implicitly through the daily unsupervised updating that already takes place in the uNN model, and thus entail minimal computational requirements on top of the regular start-up procedure. Deep neural networks with unsupervised updates can perform well over time with a fraction of the training sessions used here, which would result in more similar computational requirements for the retraining and masking approaches. However, considering the near-equivalence of the uNN-RETRAIN and the uNN-MASK when damage was simulated in up to 20 of the most important channels, the performance benefits from retraining are expected to be with minimal when the decoder is retrained with only a small number of trials. Furthermore, the entire process of identifying and masking disrupted channels requires no explicit input from either the user or a technician and is thus aligned with user preferences that no intervention is required after the initial training period.

### 7.3 Examples

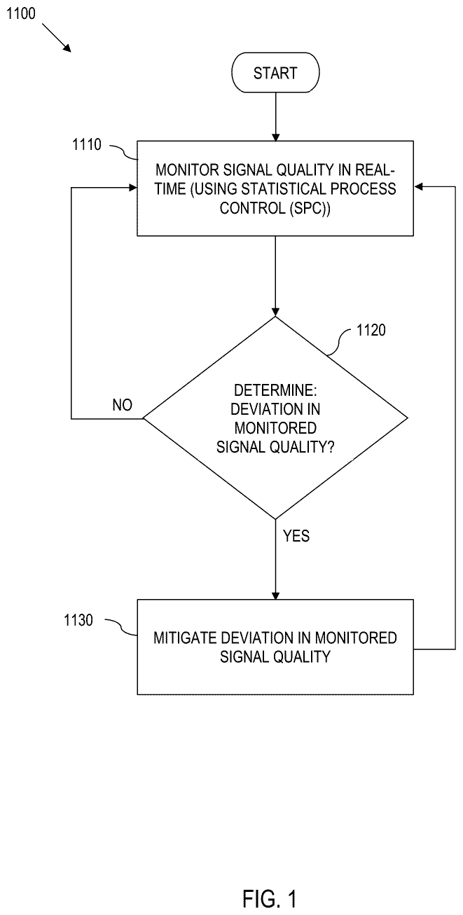

Having described in detail several embodiments that compensate for disruptions, attention is turned to FIGS. 1 through 13, which show an example embodiment of a sensor compensation process in which the mechanisms described herein, are employed.

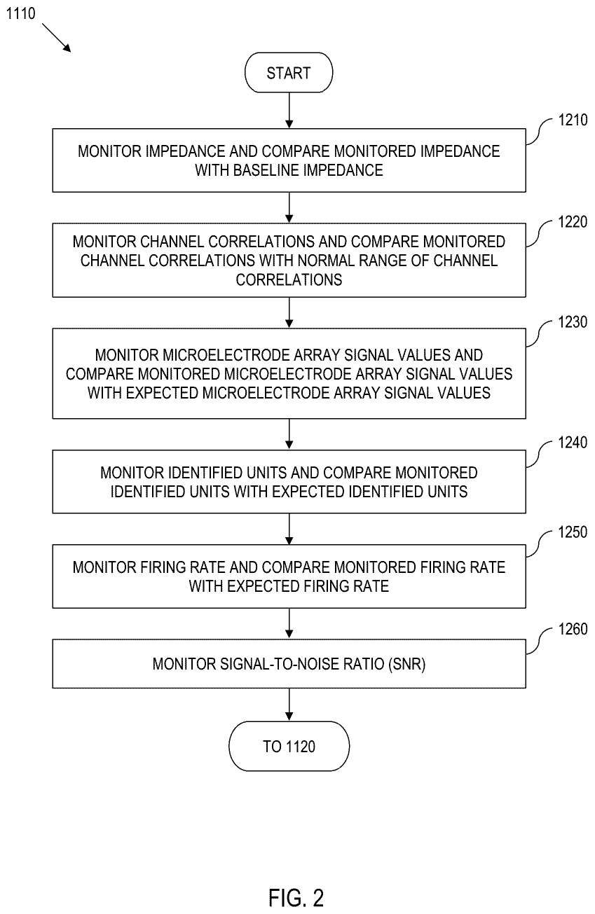

FIG. 1 is a flowchart showing an embodiment of a sensor compensation process 1100. As shown in FIG. 1, an embodiment of the process is broadly seen as comprising monitoring 1110 signal quality in real-time. Preferably, the monitoring 1110 of signal quality uses statistical process control (SPC), as described in Section 5.1, herein. The process 1100 next determines 1120 whether or not there is a deviation in the monitored signal quality. If there is no deviation, then the process 1100 continues to monitor 1110 the signal quality. If, however, there is a deviation, then the process 1100 mitigates 1130 for the deviation in the monitored signal quality. Several embodiments of the broad process 1100 are described below with reference to FIGS. 2 through 13.

FIG. 2 is a flowchart showing an embodiment of a monitoring process 1110 (FIG. 1). As shown in FIG. 2, the monitoring process 1110 comprises monitoring 1210 impedance and comparing the monitored impedance with a baseline impedance. Additionally, the monitoring process 1110 comprises monitoring 1220 channel correlations and comparing the monitored channel correlations with a normal range of channel correlations. Additionally, the monitoring process 1110 comprises monitoring 1230 microelectrode array signal values and comparing the monitored microelectrode array signal values with expected microelectrode array signal values. Additionally, the monitoring process 1110 comprises monitoring 1240 identified units and comparing the monitored identified units with expected identified units. Additionally, the monitoring process 1110 comprises monitoring 1250 firing rate and comparing the monitored firing rate with an expected firing rate. Furthermore, the monitoring process 1110 comprises monitoring 1260 signal-to-noise ratio (SNR). As discussed in Section 5.1, herein, these monitored values provide an indication of whether or not there are disruptions in the brain-machine interface (BMI).

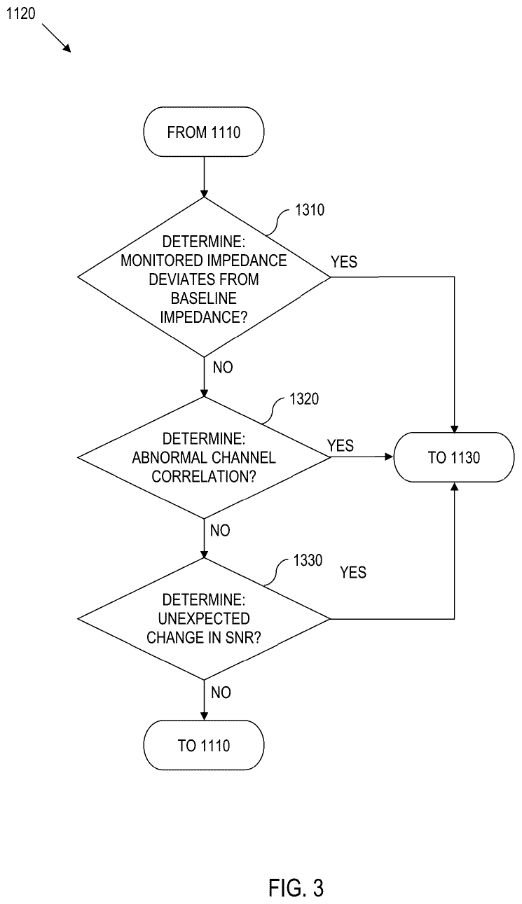

The process 1110 of FIG. 2 continues to FIG. 3, which shows a flowchart of an embodiment of a disruption determining process 1120 (FIG. 1). As shown in FIG. 3, the determining 1120 process comprises determining 1310 whether or not the monitored impedance deviates from the baseline impedance. If the monitored impedance deviates from the baseline impedance, then the process 1120 continues to mitigation 1130 (FIG. 1). If, however, there is no deviation, then the process 1120 further continues by determining 1320 whether or not there are one or more abnormal channel correlations. If there are abnormal channel correlations, then the process 1120 again contuse to mitigation 1130 (FIG. 1). If the channel correlations are normal, however, then the process continues by determining 1330 whether or not there are unexpected changes in SNR. If the SNR exhibits unexpected changes, then the process 1120 once again continues to mitigation 1130 (FIG. 1). However, if there are no unexpected changes in SNR, then the process 1120 continues to monitor 1110 (FIG. 1) signal quality. The process 1120 (FIG. 3) can be implemented in accordance with Section 5.1, herein.

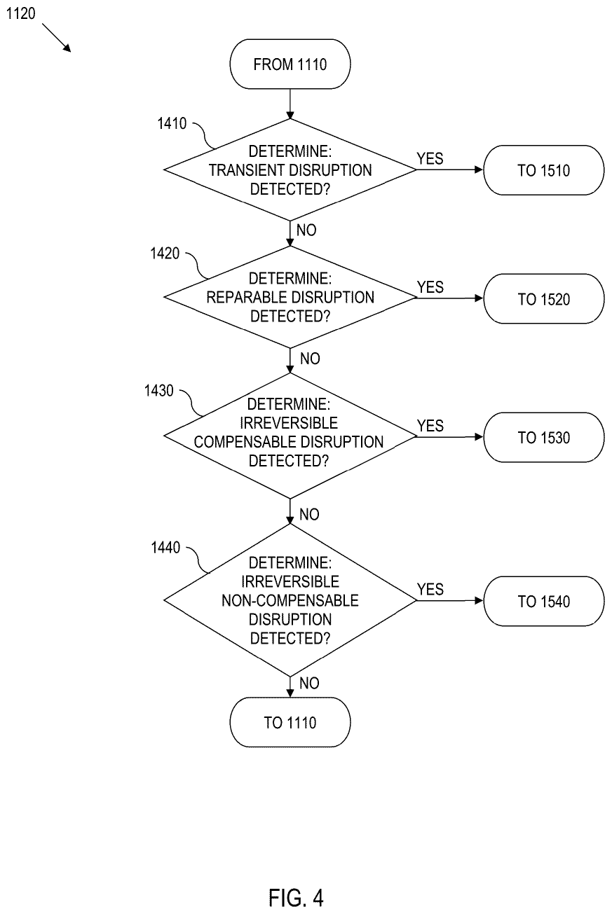

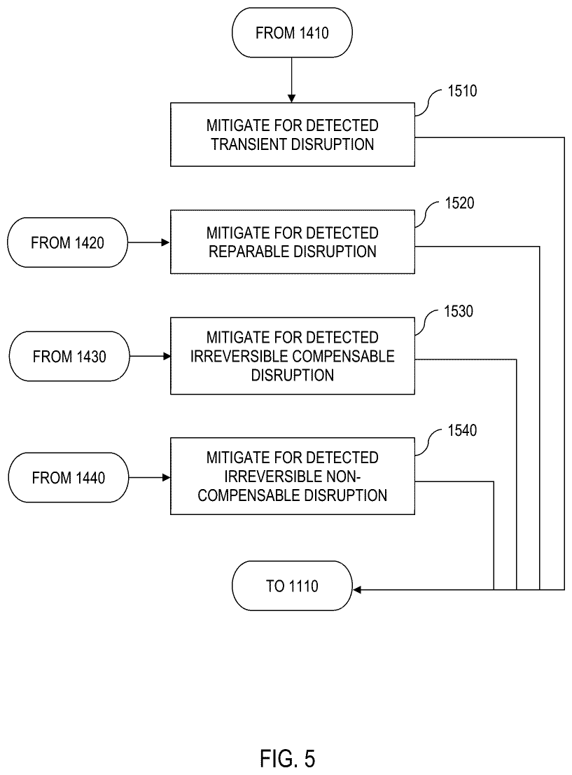

FIG. 4 is a flowchart showing another embodiment of a disruption determining process 1120 (FIG. 1). Correspondingly, FIG. 5 is a flowchart showing an embodiment of a mitigation process for the disruptions determined in FIG. 4. As shown in FIG. 4, the process 1120 comprises four (4) separate determinations, namely: determining 1410 whether or not a transient disruption is detected; determining 1420 whether or not a reparable disruption is detected; determining 1430 whether or not an irreversible compensable disruption is detected; and determining 1440 whether or not an irreversible non-compensable disruption is detected. Again, those having skill in the art will appreciate that the process 1120 (FIG. 4) can be implemented in accordance with Section 5.1, herein. With this in mind, if the process 1120 determines 1410 that there is a transient disruption, then the process continues by mitigating 1510 for the detected transient disruption, as shown in FIG. 5.

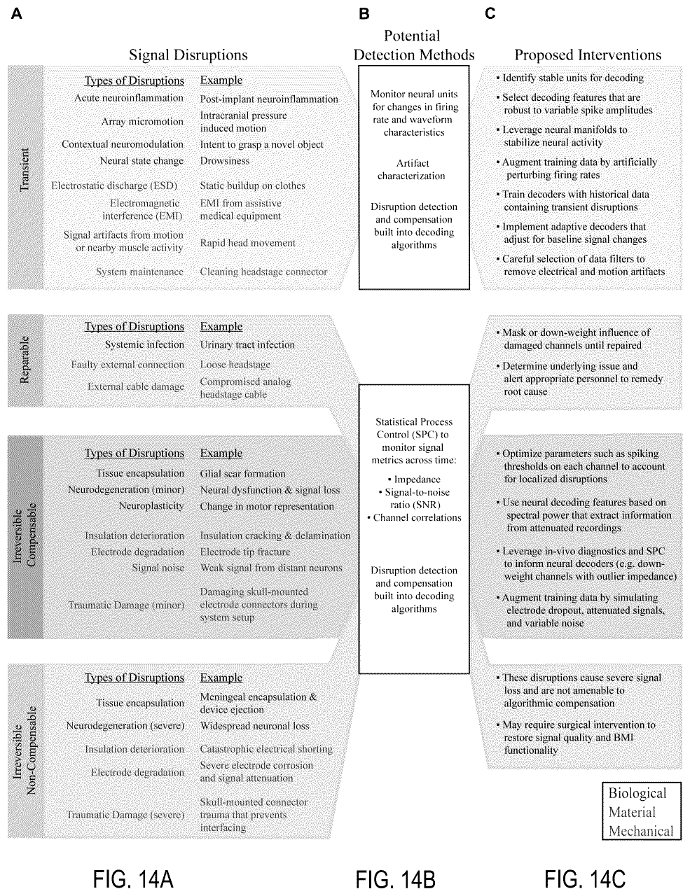

If, however, the process 1120 determines 1410 that there is no transient disruption, then the process 1120 continues by determining 1420 whether or not a reparable disruption is detected. If a reparable disruption is detected, then the process continues by mitigating 1520 for the detected reparable disruption. If, however, the process 1120 determines 1420 that there are no reparable disruptions, then the process 1120 continues by determining 1430 whether or not an irreversible compensable disruption is detected. If an irreversible compensable disruption is detected, then the process 1120 continues by mitigating 1530 for the irreversible compensable disruption. Finally, if the process 1120 determines 1430 that no irreversible compensable disruption is detected, then the process 1120 continues by determining 1440 whether or not an irreversible non-compensable disruption is detected. If an irreversible non-compensable disruption is detected, then the process 1120 continues by mitigating 1540 for the irreversible non-compensable disruption (if possible). If no disruptions are detected by the end of the process 1120 in FIG. 14, then the process 1120 returns to monitoring 1110 signal quality.

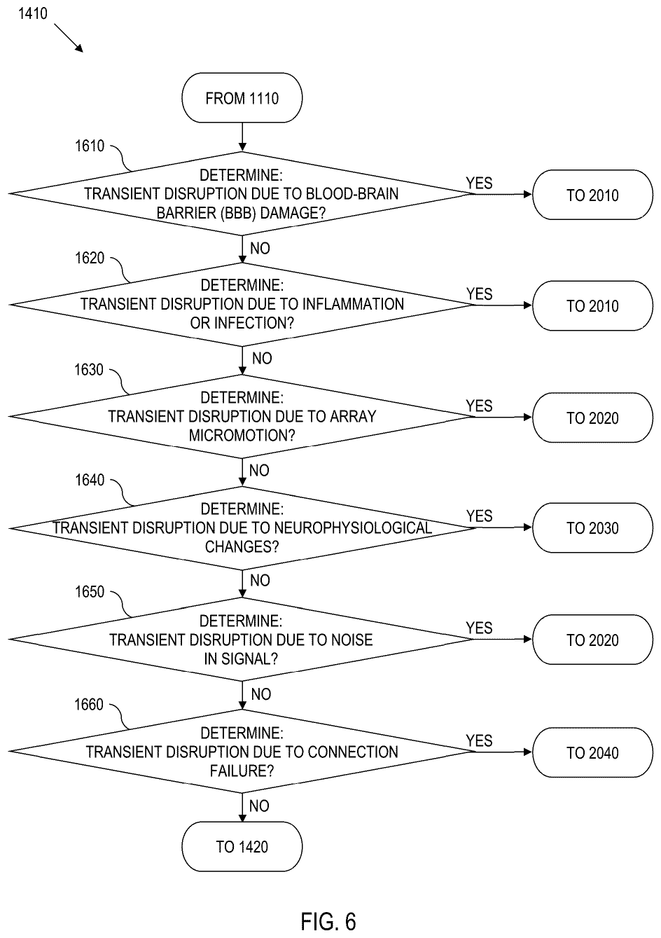

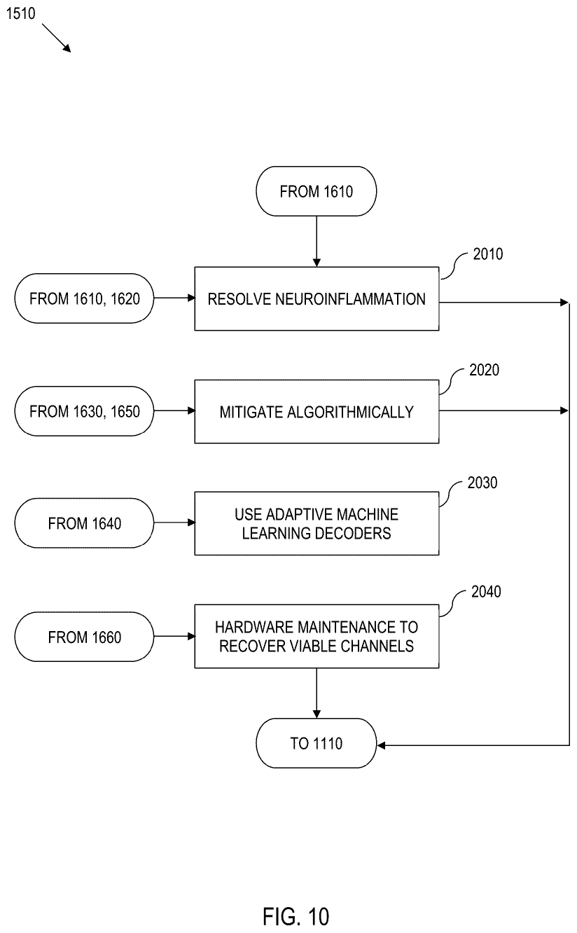

Continuing, FIG. 6 is a flowchart showing an embodiment of a transient disruption determining process 1410 as shown in FIG. 4. FIG. 10 is a flowchart showing an embodiment of a transient disruption mitigation process 1510 (FIG. 5) for the transient disruptions of FIG. 6. Notably, the processes 1410, 1510 can be implemented in accordance with the description, e.g., in Sections 2.1, 2.2, 2.3, 2.4, 2.5, 2.6, and 5.2, herein. With this in mind, the transient disruption determining process 1410 comprises determining 1610 whether or not the transient disruption is due to blood-brain barrier (BBB) damage. If BBB damage is the cause of the transient disruption, then the process 1410 continues to FIG. 10 and resolves 2010 (to the extent possible) any neuroinflammation that is the cause of the BBB-related transient disruption. If, however, the transient disruption is not due to BBB damage, then the process 1410 determines 1620 whether or not the transient disruption is due to an inflammation or an infection. If inflammation or infection is the cause of the transient disruption, then the process continues to FIG. 10 and resolves 2010 the neuroinflammation. If, however, the transient disruption is not due to inflammation or infection, then the process 1410 determines 1630 whether or not the transient disruption is due to array micromotion. If array micromotion is the cause of the transient disruption, then the process continues to FIG. 10 and mitigates 2020 the disruption algorithmically, as described in Section 5.2, herein.

In some embodiments, process 2010 “Resolve neuroinflammation” implies something that can be repaired, and thus a reparable disruption. In other embodiments, as noted more fully in section 5.2, these may be treated analogous to algorithmic mitigations for transient disruptions. Notably, some disruptions may be a “combination” of these classes and can be both transient and reparable.

If, however, the transient disruption is not due to array micromotion, then the process 1410 determines 1640 whether or not the transient disruption is due to neurophysiological changes. If neurophysiological changes are the cause of the transient disruption, then the process continues to FIG. 10 and uses 2030 adaptive machine learning decoders to compensate for the neurophysiological changes. If, however, the transient disruption is not due to neurophysiological changes, then the process 1410 determines 1650 whether or not the transient disruption is due to noise in the signal. If signal noise is the cause of the transient disruption, then the process continues to FIG. 10 and resolves mitigates 2020 the disruption algorithmically, as described in Section 5.2, herein. If, however, the transient disruption is not due to signal noise, then the process 1410 determines 1660 whether or not the transient disruption is due to a connection failure. If a connection failure is the cause of the transient disruption, then the process continues to FIG. 10 with hardware maintenance 2040 to recover viable channels. Thereafter, the process 1510 returns to monitoring 1110 (FIG. 1) signal quality.

In some embodiments, process 2040 implies something that can be repaired, and thus a reparable disruption. In other embodiments, as noted more fully in section 5.2, these may be treated analogous to algorithmic mitigations for transient disruptions. Notably, some disruptions may be a “combination” of these classes and can be both transient and reparable.

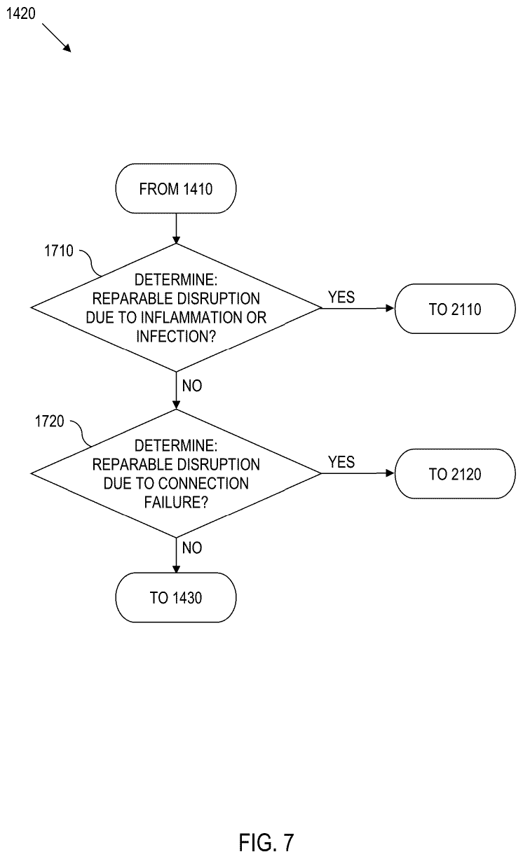

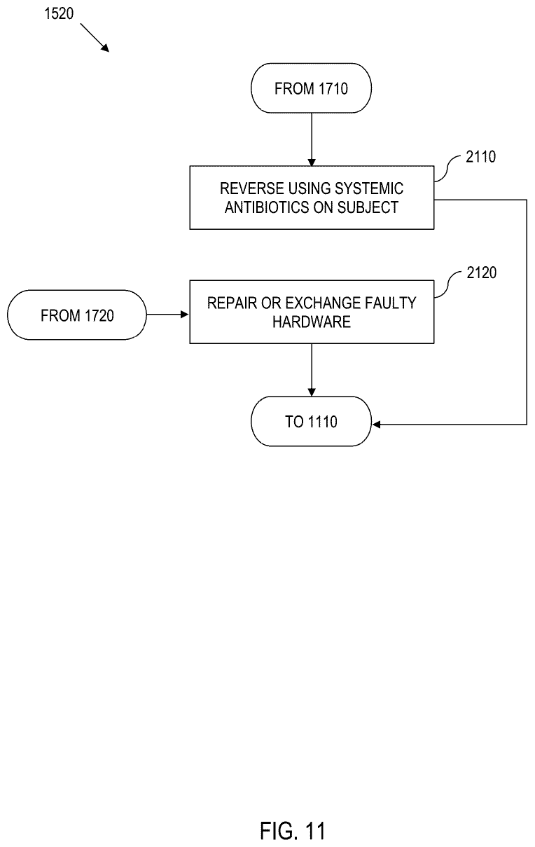

Continuing, FIG. 7 is a flowchart showing an embodiment of a reparable disruption determining process 1420 as shown in FIG. 4. FIG. 11 is a flowchart showing an embodiment of a reparable disruption mitigation process 1520 (FIG. 5) for the reparable disruptions of FIG. 7. The processes 1420, 1520 can be implemented in accordance with the description, e.g., in Sections 2.4, 4.2 and 5.3, herein. With this in mind, the reparable disruption determining process 1420 comprises determining 1710 whether or not the reparable disruption is due to an inflammation or an infection. If inflammation or infection is the cause of the reparable disruption, then the process continues to FIG. 11 and attempts to reverse 2110 the disruption through use of systemic antibiotics on the subject (or patient). If, however, the reparable disruption is not due to inflammation or infection, then the process 1420 determines 1720 whether or not the reparable disruption is due to a connection failure. If a connection failure is the cause of the reparable disruption, then the process continues to FIG. 11 with faulty hardware being repaired or replaced 2120. Thereafter, the process 1520 returns to monitoring 1110 (FIG. 1) signal quality.

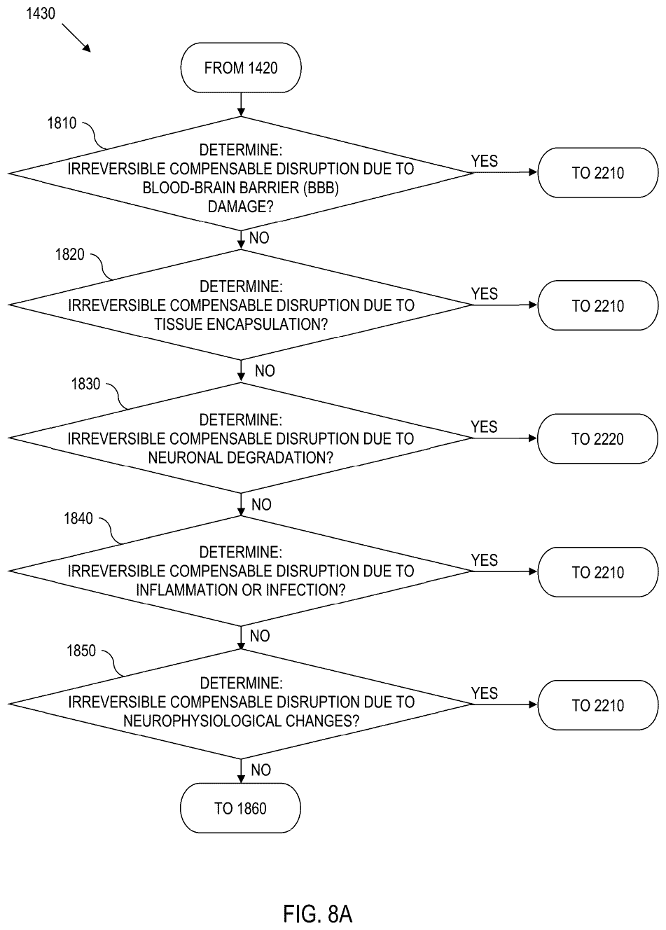

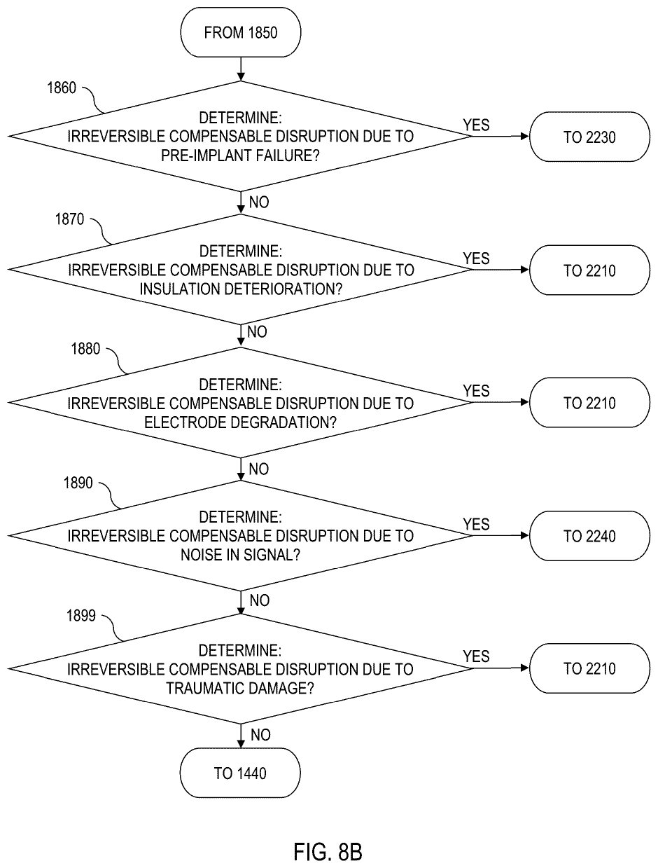

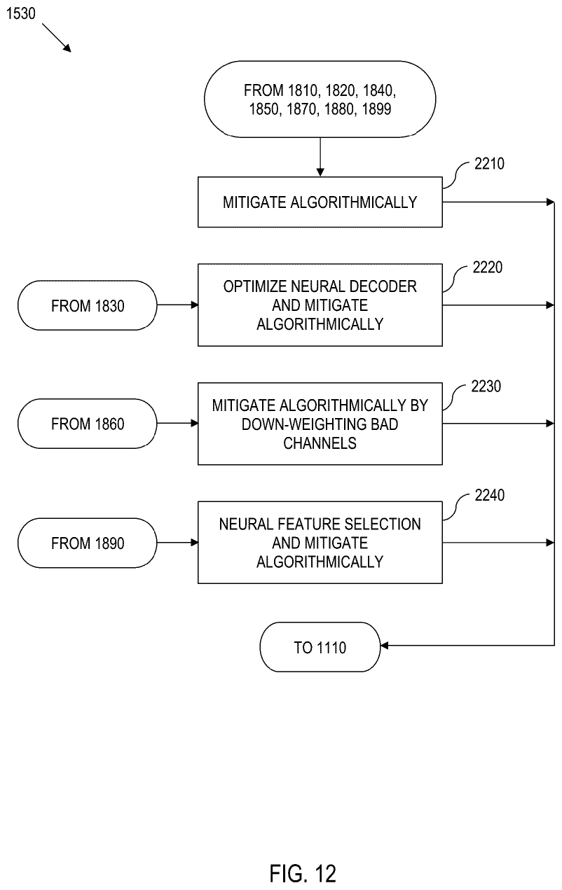

Continuing, FIGS. 8A and 8B are flowcharts showing an embodiment of an irreversible compensable disruption determining process 1430 as shown in FIG. 4. FIG. 12 is a flowchart showing an embodiment of an irreversible compensable disruption mitigation process 1530 for the irreversible compensable disruptions of FIGS. 8A and 8B. The processes 1430, 1530 can be implemented in accordance with the description, e.g., in Sections 2.1, 2.2, 2.3, 2.4, 2.5, 2.6, 3.1, 3.2, 3.3, 4.1, 4.2, and 5.4, herein. With this in mind, the irreversible compensable disruption determining process 1430 comprises determining 1810 whether or not the irreversible compensable disruption is due to blood-brain barrier (BBB) damage. If BBB damage is the cause of the irreversible compensable disruption, then the process 1430 continues to FIG. 2 and mitigates 2210 (to the extent possible) the irreversible compensable disruption algorithmically. If, however, the irreversible compensable disruption is not due to BBB damage, then the process 1430 determines 1820 whether or not the irreversible compensable disruption is due to tissue encapsulation.

If tissue encapsulation is the cause of the irreversible compensable disruption, then the process continues to FIG. 12 and again mitigates 2210 algorithmically. If, however, the irreversible compensable disruption is not due to tissue encapsulation, then the process 1430 determines 1830 whether or not the irreversible compensable disruption is due to neuronal degeneration. If neuronal degeneration is the cause of the irreversible compensable disruption, then the process continues to FIG. 2 and optimizes 2220 neural decoders and mitigates algorithmically, as described in Section 5.4, herein. If, however, the irreversible compensable disruption is not due to neuronal degeneration, then the process 1430 determines 1840 whether or not the irreversible compensable disruption is due to an inflammation or an infection. If either an inflammation or an infection (or both) is the cause of the irreversible compensable disruption, then the process continues to FIG. 12 and once again mitigates 2210 algorithmically (see Section 5.4). If, however, the irreversible compensable disruption is not due to inflammation or infection, then the process 1430 determines 1850 whether or not the irreversible compensable disruption is due to neurophysiological changes. If neurophysiological changes are the cause of the irreversible compensable disruption, then the process continues to FIG. 12 and once again mitigates 2210 the disruption algorithmically, as described in Section 5.4, herein. If, however, the irreversible compensable disruption is not due to neurophysiological changes, then the process 1430 continues to FIG. 8B, where it determines 1860 whether or not the irreversible compensable disruption is due to a pre-implant failure.

If a pre-implant failure is the cause of the irreversible compensable disruption, then the process continues to FIG. 12 and the process 1530 mitigates 2230 algorithmically by down-weighting bad channels. If a pre-implant failure is not the cause of the irreversible compensable disruption, then the process determines 1870 whether or not the irreversible compensable disruption is due to insulation deterioration. If insulation deterioration is the cause of the irreversible compensable disruption, then the process continues to FIG. 12 and the process 1530 once again mitigates 2210 algorithmically. If insulation deterioration is not the cause of the irreversible compensable disruption, then the process determines 1880 whether or not the irreversible compensable disruption is due to electrode degradation/degeneration.

If electrode degradation/degeneration is the cause of the irreversible compensable disruption, then the process continues to FIG. 12 and the process 1530 once again mitigates 2210 algorithmically. If electrode degradation/degeneration is not the cause of the irreversible compensable disruption, then the process determines 1890 whether or not the irreversible compensable disruption is due to noise in the signal. If signal noise is the cause of the irreversible compensable disruption, then the process continues to FIG. 12 and the process 1530 mitigates 2240 by judicious selection of neural features and, also, mitigating algorithmically, as described in Section 5.4, herein. If signal noise is not the cause of the irreversible compensable disruption, then the process determines 1899 whether or not the irreversible compensable disruption is due to traumatic damage. If traumatic damage is the cause of the irreversible compensable disruption, then the process continues to FIG. 12 and the process 1530 once again mitigates 2210 algorithmically, as described in Section 5.4, herein. Thereafter, the process 1530 returns to monitoring 1110 (FIG. 1) signal quality.

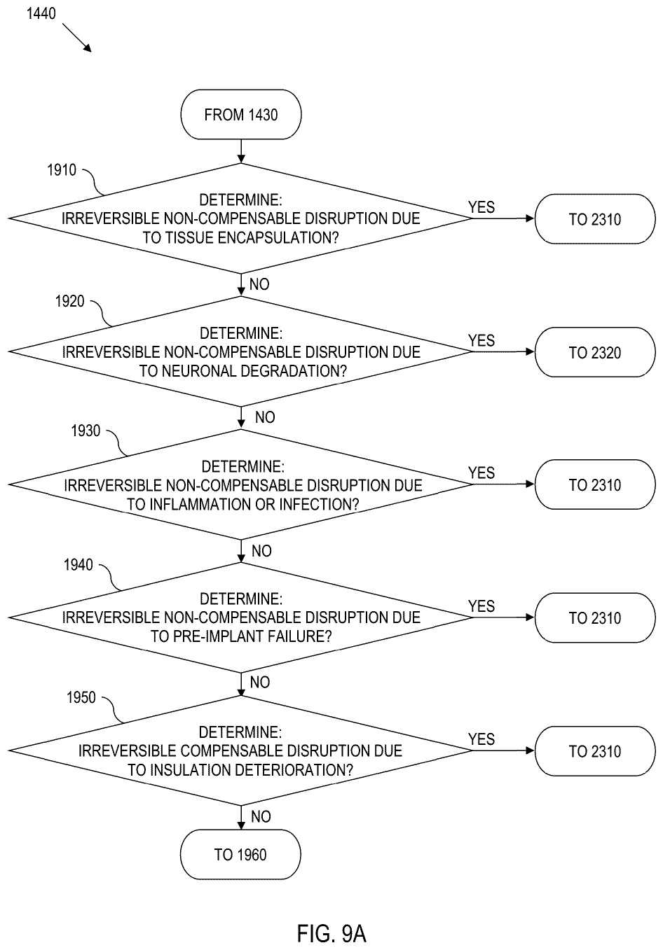

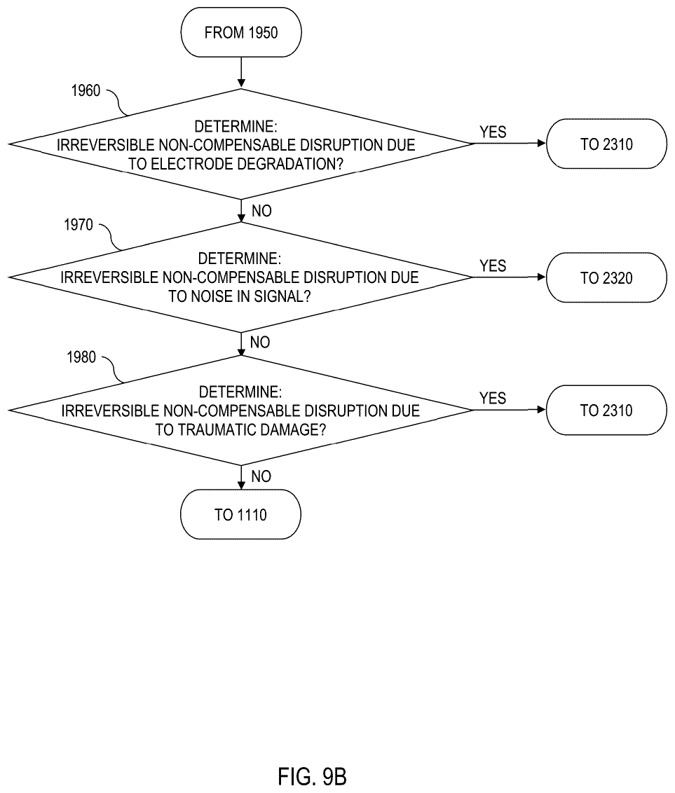

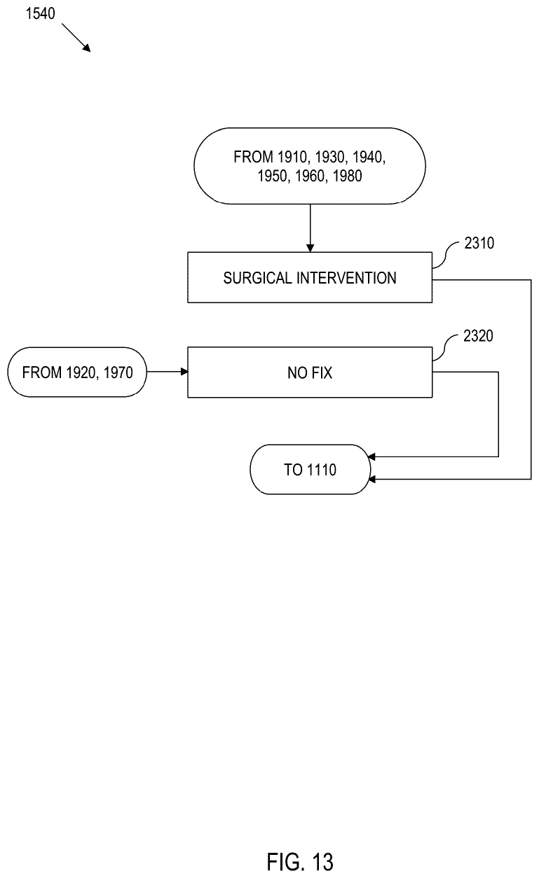

Continuing, FIGS. 9A and 9B are flowcharts showing an embodiment of an irreversible non-compensable disruption determining process 1440 as shown in FIG. 4. FIG. 13 is a flowchart showing an embodiment of an irreversible non-compensable disruption mitigation process 1540 (FIG. 5) for the irreversible non-compensable disruptions of FIGS. 9A and 9B. To the extent that an irreversible non-compensable disruption can be fixed at all, the processes 1440, 1540 can be implemented in accordance with the description, e.g., in Sections 2.1, 2.2, 2.3, 2.4, 2.5, 2.6, 3.1, 3.2, 3.3, 4.1, and 4.2 herein.

With this in mind, the irreversible non-compensable disruption determining process 1440 comprises determining 1910 whether or not the irreversible non-compensable disruption is due to tissue encapsulation. If tissue encapsulation is the cause of the irreversible non-compensable disruption, then the process continues to FIG. 13, which requires surgical intervention 2310. If, however, the irreversible non-compensable disruption is not due to tissue encapsulation, then the process 1440 determines 1920 whether or not the irreversible non-compensable disruption is due to neuronal degeneration. If neuronal degeneration is the cause of the irreversible non-compensable disruption, then the process continues to FIG. 13, which shows that there is no fix for neuronal degeneration. Thereafter, the process 1540 returns to monitoring 1110 (FIG. 1) signal quality. If the irreversible non-compensable disruption is not due to neuronal degeneration, then the process 1440 determines 1930 whether or not the irreversible non-compensable disruption is due to an inflammation or an infection.

Notably, in some embodiments, encapsulation issues may fall on a continuous spectrum. For instance, there may be no fix for meningeal tissue encapsulation or neuronal degeneration, as they progress to severe levels until eventually, a signal is unrecoverable. In this case, surgical intervention becomes a solution. In early stages it may be that there is “no fix” but does not warrant brain surgery due to the inherent risks. Accordingly, in some embodiments, such conditions need not follow different paths, e.g., as illustrated. Rather, there are situations where flow in the flow charts of FIGS. 1-13. It can be case-specific. In some embodiments, if neuronal degeneration is bad enough where it can't be compensated for (irreversible non compensable), it may optionally follow the same path requiring surgical removal.

If either an inflammation or an infection (or both) is the cause of the irreversible non-compensable disruption, then the process continues to FIG. 13 and once surgical intervention 2310 is required. If, however, the irreversible non-compensable disruption is not due to inflammation or infection, then the process 1440 determines 1940 whether or not the irreversible non-compensable disruption is due to a pre-implant failure. If a pre-implant failure is the cause of the irreversible non-compensable disruption, then the process continues to FIG. 13 and the process 1540 again requires surgical intervention 2310. If a pre-implant failure is not the cause of the irreversible non-compensable disruption, then the process determines 1950 whether or not the irreversible non-compensable disruption is due to insulation deterioration. If insulation deterioration is the cause of the irreversible non-compensable disruption, then the process continues to FIG. 13 and the process 1540 once again requires surgical intervention 2310.

If insulation deterioration is not the cause of the irreversible non-compensable disruption, then the process continues to FIG. 9B and determines 1960 whether or not the irreversible non-compensable disruption is due to electrode degradation/degeneration. If electrode degradation/degeneration is the cause of the irreversible non-compensable disruption, then the process continues to FIG. 13 and the process 1540 once again requires surgical intervention 2310. If electrode degradation/degeneration is not the cause of the irreversible non-compensable disruption, then the process determines 1970 whether or not the irreversible non-compensable disruption is due to noise in the signal. If signal noise is the cause of the irreversible non-compensable disruption, then the process continues to FIG. 13, which shows that the problem cannot be fixed 2320. If signal noise is not the cause of the irreversible non-compensable disruption, then the process determines 1999 whether or not the irreversible non-compensable disruption is due to traumatic damage.

If traumatic damage is the cause of the irreversible non-compensable disruption, then the process continues to FIG. 3 and the process 1540 once again requires surgical intervention 2310. Thereafter, the process 1540 returns to monitoring 1110 (FIG. 1) signal quality.

As shown in the embodiments of FIGS. 1 through 13, the particular mitigation process is determined based on the particular type of disruption. Thus, rather than addressing only the cause of the disruption, the processes herein further address the disruptive effect, thereby permitting compensation at the effect (rather than solely at the cause). By compensating the effect, the various embodiments allow for better mitigation when disruptions occur at the BMI.

Example Signal Disruption Classification

An overview of signal disruptions with their expanded classifications, potential detection methods, and compensatory strategies is shown in FIG. 14A-14C.

Referring to FIG. 14A, signal disruptions are classified according to their underlying cause (Biological, Material, or Mechanical), and impact on signal quality and responsiveness to intervention (Transient, Reparable, Irreversible Compensable, and Irreversible Non-Compensable).

Referring to FIG. 14B, signal disruptions can be explicitly detected with statistical monitoring of neural features and recording metrics. Following the detection of a disruption, BMIs can initiate tailored algorithmic countermeasures to adapt to changes in signal characteristics. In parallel, advanced machine learning algorithms and decoder training strategies mitigate the effect of disruptions without requiring explicit detection.

Referring to FIG. 14C, disruption classes herein have characteristic interventions that help maintain BMI performance. Signal preprocessing, data augmentation, neural feature selection, neural manifolds, and adaptive neural decoders are among the most useful techniques for mitigating the effects of recording disruptions.

Miscellaneous

Referring to the FIGURES generally, MEA signals may be disrupted, e.g., by biologic tissue reactions around the electrode tips, deterioration of electrode materials, or mechanical connection failures. Detection methods herein leverage the characteristic manner in which disruptions affect recorded signals, allowing for targeted interventions to restore signal quality and BMI function.

Recording disruptions can impair motor intention decoding. By way of example, the performance of three-deep neural network decoder variants were evaluated for a four-movement motor imagery task over the span of one year. Fixed neural network decoder parameters remained unchanged for the duration of the evaluation. The other networks were updated each session with data from a preceding recording block with either explicit training labels. Both the sNN and uNN were able to adapt to daily changes in recording conditions and thus outperform the fNN.

As noted more fully herein, some disruptions may be a “combination” of classes. As such, the flowcharts herein can be modified accordingly to account for such combinations, e.g., disruptions that can be both transient and reparable. Moreover, the algorithmic strategies herein may be useful for multiple disruptions within the same category. For example, process 1640 to account for neurophysiological changes would likely benefit from the decoder training strategies and data augmentation covered herein, in addition to the adaptive machine learning models (e.g., process 2030). Another example is process 1820->2210 (FIG. 8) which may benefit from neural feature selection (process 2240). Furthermore, in some embodiments, 1870 may benefit from 2230 and 2240, etc. There are many other instances here where an algorithmic intervention is broadly applicable to many of the disruptions within the same class, and thus the above is presented by way of illustration, and not by way of limitation.

### Variants

As discussed above, the classification of disruptions can help identify a solution to mask channels or update software within the human-machine interface (e.g., BMI) to compensate for the disruption event. Further, in some cases, when a disruption event is detected, a warning may be issued to the user, to a technician, or both to indicate that the user should stop using the device immediately.

The processes described herein may be implemented in hardware, software, firmware, or a combination thereof. In the preferred embodiment(s), the processes are implemented in software or firmware that is stored in a memory and that is executed by a suitable instruction execution system. If implemented in hardware, as in an alternative embodiment, the processes can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Any process descriptions or blocks in flow charts should be understood as representing modules, segments, or portions of code which include one or more executable instructions for implementing specific logical functions or steps in the process, and alternate implementations are included within the scope of the preferred embodiment of the present disclosure in which functions may be executed out of order from that shown or discussed, including substantially concurrently or in reverse order, depending on the functionality involved.

The processes described herein are executable as a computer program, which comprises an ordered listing of executable instructions for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can contain, store, communicate, propagate, or transport the program for use by or in connection with the instruction execution system, apparatus, or device. The computer-readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical). Note that the computer-readable medium could even be paper or another suitable medium upon which the program is printed, as the program can be electronically captured via, for instance, optical scanning of the paper or other medium, then compiled, interpreted or otherwise processed in a suitable manner if necessary, and then stored in a computer memory.

Although exemplary embodiments have been shown and described, it will be clear to those of ordinary skill in the art that a number of changes, modifications, or alterations to the disclosure as described may be made. All such changes, modifications, and alterations should therefore be seen as within the scope of the disclosure.

What is claimed is:

1. A process for compensating for disruptions at a human-machine interface, the process comprising:
monitoring signal quality;
determining deviations in the monitored signal quality; and
mitigating for the determined deviations.

2. The process of claim 1, wherein monitoring signal quality comprises monitoring signal quality in real-time.

3. The process of claim 2, wherein monitoring signal quality comprises using statistical process control (SPC).

4. The process of claim 3, wherein monitoring signal quality comprises:
monitoring impedance;
monitoring channel correlations;
monitoring microelectrode array signal values;
monitoring identified units;
monitoring firing rate; comparing the monitored impedance with a baseline impedance;
comparing the monitored channel correlations with a normal range of channel correlations;
comparing the monitored microelectrode array signal values with expected monitored microelectrode array signal values;
comparing the monitored identified units with expected identified units;
comparing the monitored firing rate with an expected firing rate; and
monitoring signal-to-noise ratio (SNR).

5. The process of claim 4, wherein determining deviations comprises:
determining whether or not the monitored impedance deviates from a baseline impedance;
determining whether or not there are abnormal microelectrode array signal values;
determining whether or not there are abnormal identified units;
determining whether or not there is an abnormal firing rate;
determining whether or not there is an abnormal channel correlations; and
determining whether or not there is an unexpected change in SNR.

6. The process of claim 1, wherein monitoring signal quality comprises monitoring signal quality while a user of the human-machine interface is at rest.

7. The process of claim 1, wherein monitoring signal quality comprises monitoring signal quality while a user of the human-machine interface is performing a motor task.

8. The process of claim 1, wherein mitigating for the determined deviations further comprises:
masking channels experiencing the determined deviations; and
updating, automatically, a model to reassign weights of channels not masked.

9. The process of claim 1, wherein mitigating for the determined deviations further comprises issuing a warning for a user to stop use of the human-machine interface.

10. A process for compensating for disruptions at a human-machine interface, the process comprising:
monitoring signal quality;
determining whether or not a transient disruption is detected in the monitored signal quality;
mitigating, in response to determining that a transient disruption is detected, for the transient disruption;
determining whether or not a reparable disruption is detected in the monitored signal quality;
mitigating, in response to determining that a reparable disruption is detected, for the reparable disruption;
determining whether or not an irreversible compensable disruption is detected in the monitored signal quality;
mitigating, in response to determining that an irreversible compensable disruption is detected, for the irreversible compensable disruption;
determining whether or not an irreversible non-compensable disruption is detected in the monitored signal quality; and
mitigating, to the extent possible, in response to determining that an irreversible non-compensable disruption is detected, for the irreversible non-compensable disruption.

11. The process of claim 10, wherein monitoring signal quality comprises monitoring signal quality in real-time.

12. The process of claim 10, wherein determining whether or not the transient disruption is detected comprises:
determining whether or not the transient disruption is due to damage in a blood-brain barrier (BBB);
determining whether or not the transient disruption is due to an inflammation;
determining whether or not the transient disruption is due to an infection;
determining whether or not the transient disruption is due to array micromotion;
determining whether or not the transient disruption is due to a neurophysiological change;
determining whether or not the transient disruption is due to signal noise;
determining whether or not the transient disruption is due to a connection failure; or
a combination thereof.

13. The process of claim 10, wherein mitigating for the transient disruption comprises:
resolving a neuroinflammation;
mitigating for the disruption algorithmically;
maintaining hardware to recover viable channels; or
a combination thereof.

14. The process of claim 13, wherein:
mitigating for the disruption algorithmically comprises using adaptive machine learning decoders.

15. The process of claim 10, wherein determining whether or not the irreversible compensable disruption is detected comprises:
determining whether or not the irreversible compensable disruption is due to blood-brain barrier (BBB) damage;
determining whether or not the irreversible compensable disruption is due to tissue encapsulation;
determining whether or not the irreversible compensable disruption is due to neuronal degeneration;
determining whether or not the irreversible compensable disruption is due to inflammation;
determining whether or not the irreversible compensable disruption is due to infection;
determining whether or not the irreversible compensable disruption is due to a neurophysiological change;
determining whether or not the irreversible compensable disruption is due to a pre-implant failure;
determining whether or not the irreversible compensable disruption is due to insulation deterioration;
determining whether or not the irreversible compensable disruption is due to electrode degradation/degeneration;
determining whether or not the irreversible compensable disruption is due to signal noise;
determining whether or not the irreversible compensable disruption is due to traumatic damage; or
a combination thereof.

16. The process of claim 15, wherein mitigating for the irreversible compensable disruption comprises:
mitigating the irreversible compensable disruption algorithmically;
optimizing neural decoders and mitigating the irreversible compensable disruption algorithmically;
mitigating the irreversible compensable disruption algorithmically by down-weighting bad channels;
judiciously selecting neural features and mitigating algorithmically; or
a combination thereof.

17. The process of claim 10, wherein determining whether or not the irreversible non-compensable disruption is detected comprises:
determining whether or not the irreversible non-compensable disruption is due to tissue encapsulation;
determining whether or not the irreversible non-compensable disruption is due to neuronal degeneration;
determining whether or not the irreversible non-compensable disruption is due to inflammation;
determining whether or not the irreversible non-compensable disruption is due to infection;
determining whether or not the irreversible non-compensable disruption is due to a pre-implant failure;
determining whether or not the irreversible non-compensable disruption is due to insulation deterioration;
determining whether or not the irreversible non-compensable disruption is due to electrode degradation/degeneration;
determining whether or not the irreversible non-compensable disruption is due to signal noise;
determining whether or not the irreversible non-compensable disruption is due to traumatic damage; or
a combination thereof.

18. The process of claim 17, wherein mitigating for the irreversible non-compensable disruption comprises:
requiring surgical intervention; or
determining that there is no fix to the irreversible non-compensable disruption.

19. A process for compensating for disruptions at a human-machine interface, the process comprising:
monitoring signal quality;
determining whether or not a transient disruption is detected in the monitored signal quality, wherein determining whether or not the transient disruption is detected comprises:
determining whether or not the transient disruption is due to damage in a blood-brain barrier (BBB);
determining whether or not the transient disruption is due to an inflammation;
determining whether or not the transient disruption is due to an infection;
determining whether or not the transient disruption is due to array micromotion;
determining whether or not the transient disruption is due to a neurophysiological change;
determining whether or not the transient disruption is due to signal noise;
determining whether or not the transient disruption is due to a connection failure; or
a combination thereof;
mitigating, in response to determining that a transient disruption is detected, for the transient disruption, wherein mitigating for the transient disruption comprises:
resolving a neuroinflammation;
mitigating for the disruption algorithmically;
using adaptive machine learning decoders;
maintaining hardware to recover viable channels; or
a combination thereof;
determining whether or not a reparable disruption is detected in the monitored signal quality, wherein determining whether or not the reparable disruption is detected comprises:
determining whether or not the reparable disruption is due to an inflammation;
determining whether or not the reparable disruption is due to an infection;
determining whether or not the reparable disruption is due to a connection failure; or
a combination thereof;
mitigating, in response to determining that a reparable disruption is detected, for the reparable disruption, wherein mitigating for the reparable disruption comprises:
reversing the reparable disruption using systemic antibiotics on a subject or patient;
repairing or exchanging faulty hardware; or
a combination thereof;
determining whether or not an irreversible compensable disruption is detected in the monitored signal quality, wherein determining whether or not the irreversible compensable disruption is detected comprises:
determining whether or not the irreversible compensable disruption is due to blood-brain barrier (BBB) damage;
determining whether or not the irreversible compensable disruption is due to tissue encapsulation;
determining whether or not the irreversible compensable disruption is due to neuronal degeneration;
determining whether or not the irreversible compensable disruption is due to inflammation;
determining whether or not the irreversible compensable disruption is due to infection;
determining whether or not the irreversible compensable disruption is due to a neurophysiological change;
determining whether or not the irreversible compensable disruption is due to a pre-implant failure;
determining whether or not the irreversible compensable disruption is due to insulation deterioration;
determining whether or not the irreversible compensable disruption is due to electrode degradation/degeneration;
determining whether or not the irreversible compensable disruption is due to signal noise;
determining whether or not the irreversible compensable disruption is due to traumatic damage; or
a combination thereof;
mitigating, in response to determining that an irreversible compensable disruption is detected, for the irreversible compensable disruption, wherein mitigating for the irreversible compensable disruption comprises:
mitigating the irreversible compensable disruption algorithmically;
optimizing neural decoders and mitigating the irreversible compensable disruption algorithmically;
mitigating the irreversible compensable disruption algorithmically by down-weighting bad channels;
judiciously selecting neural features and mitigating algorithmically; or
a combination thereof;
determining whether or not an irreversible non-compensable disruption is detected in the monitored signal quality, wherein determining whether or not the irreversible non-compensable disruption is detected comprises:
determining whether or not the irreversible non-compensable disruption is due to tissue encapsulation;
determining whether or not the irreversible non-compensable disruption is due to neuronal degeneration;
determining whether or not the irreversible non-compensable disruption is due to inflammation;
determining whether or not the irreversible non-compensable disruption is due to infection;
determining whether or not the irreversible non-compensable disruption is due to a pre-implant failure;
determining whether or not the irreversible non-compensable disruption is due to insulation deterioration;
determining whether or not the irreversible non-compensable disruption is due to electrode degradation/degeneration;
determining whether or not the irreversible non-compensable disruption is due to signal noise;
determining whether or not the irreversible non-compensable disruption is due to traumatic damage; or
a combination thereof; and
mitigating, in response to determining that an irreversible non-compensable disruption is detected, for the irreversible non-compensable disruption, wherein mitigating for the irreversible non-compensable disruption comprises:
requiring surgical intervention; or
determining that there is no fix to the irreversible non-compensable disruption.

20. A process for compensating for disruptions at a human-machine interface, the process comprising:
monitoring signal quality;
determining whether or not a transient disruption is detected in the monitored signal quality, wherein determining whether or not the transient disruption is detected comprises:
determining whether or not the transient disruption is due to damage in a blood-brain barrier (BBB);
determining whether or not the transient disruption is due to an inflammation;
determining whether or not the transient disruption is due to an infection;
determining whether or not the transient disruption is due to array micromotion;
determining whether or not the transient disruption is due to a neurophysiological change;
determining whether or not the transient disruption is due to signal noise; and
determining whether or not the transient disruption is due to a connection failure;
mitigating, in response to determining that a transient disruption is detected, for the transient disruption, wherein mitigating for the transient disruption comprises:
resolving a neuroinflammation;
mitigating for the disruption algorithmically;
using adaptive machine learning decoders;
maintaining hardware to recover viable channels; or
a combination thereof;
determining whether or not a reparable disruption is detected in the monitored signal quality, wherein determining whether or not the reparable disruption is detected comprises:
determining whether or not the reparable disruption is due to an inflammation;
determining whether or not the reparable disruption is due to an infection; and
determining whether or not the reparable disruption is due to a connection failure;
mitigating, in response to determining that a reparable disruption is detected, for the reparable disruption, wherein mitigating for the reparable disruption comprises:
reversing the reparable disruption using systemic antibiotics on a subject or patient;
repairing or exchanging faulty hardware; or
a combination thereof;
determining whether or not an irreversible compensable disruption is detected in the monitored signal quality, wherein determining whether or not the irreversible compensable disruption is detected comprises:
determining whether or not the irreversible compensable disruption is due to blood-brain barrier (BBB) damage;
determining whether or not the irreversible compensable disruption is due to tissue encapsulation;
determining whether or not the irreversible compensable disruption is due to neuronal degeneration;
determining whether or not the irreversible compensable disruption is due to inflammation;
determining whether or not the irreversible compensable disruption is due to infection;
determining whether or not the irreversible compensable disruption is due to a neurophysiological change;
determining whether or not the irreversible compensable disruption is due to a pre-implant failure;
determining whether or not the irreversible compensable disruption is due to insulation deterioration;
determining whether or not the irreversible compensable disruption is due to electrode degradation/degeneration;
determining whether or not the irreversible compensable disruption is due to signal noise; and
determining whether or not the irreversible compensable disruption is due to traumatic damage;
mitigating, in response to determining that an irreversible compensable disruption is detected, for the irreversible compensable disruption, wherein mitigating for the irreversible compensable disruption comprises:
mitigating the irreversible compensable disruption algorithmically;
optimizing neural decoders and mitigating the irreversible compensable disruption algorithmically;
mitigating the irreversible compensable disruption algorithmically by down-weighting bad channels;
judiciously selecting neural features and mitigating algorithmically; or
a combination thereof;
determining whether or not an irreversible non-compensable disruption is detected in the monitored signal quality, wherein determining whether or not the irreversible non-compensable disruption is detected comprises:
determining whether or not the irreversible non-compensable disruption is due to tissue encapsulation;
determining whether or not the irreversible non-compensable disruption is due to neuronal degeneration;
determining whether or not the irreversible non-compensable disruption is due to inflammation;
determining whether or not the irreversible non-compensable disruption is due to infection;
determining whether or not the irreversible non-compensable disruption is due to a pre-implant failure;
determining whether or not the irreversible non-compensable disruption is due to insulation deterioration;
determining whether or not the irreversible non-compensable disruption is due to electrode degradation/degeneration;
determining whether or not the irreversible non-compensable disruption is due to signal noise; and
determining whether or not the irreversible non-compensable disruption is due to traumatic damage; and
mitigating, in response to determining that an irreversible non-compensable disruption is detected, for the irreversible non-compensable disruption, wherein mitigating for the irreversible non-compensable disruption comprises:
requiring surgical intervention; or
determining that there is no fix to the irreversible non-compensable disruption.
